(19) United States
(12) Patent Application Publication
(10) Pub. No.: US 2014/0235643 A1
(43) Pub. Date: Aug. 21, 2014

(54) NOVEL QUINOXALINE INHIBITORS OF PI3K

(71) Applicant: GILEAD CALISTOGA LLC, Foster City, CA (US)

(72) Inventors: Jerry B. Evarts, Redmond, WA (US); Kamal D. Puri, Lynnwood, WA (US); Roger G. Ulrich, Sammamish, WA (US); Edward A. Kesicki, Bothell, WA (US); Thomas Judge, Otsego, MI (US)

(21) Appl. No.: 14/350,039

(22) PCT Filed: Oct. 4, 2012
(86) PCT No.: PCT/US2012/058800

§ 371 Date: Apr. 4, 2014

Related U.S. Application Data
(60) Provisional application No. 61/543,176, filed on Oct. 4, 2011.

(51) Int. Cl. C07D 473/34

(52) U.S. Cl. CPC C07D 473/34
USPC 514/249; 544/277

(57) ABSTRACT

The invention provides methods that relate to a novel therapeutic strategy for the treatment of cancer and inflammatory diseases. In particular, the method comprises administration of a compound of Formula I, or a pharmaceutically acceptable salt thereof, or a pharmaceutical composition comprising such compound admixed with at least one pharmaceutically acceptable excipient.

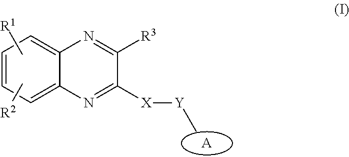

### RELATED APPLICATIONS

This application claims priority from U.S. provisional application No. 61/543,176 filed Oct. 4, 2011. The contents of these documents are incorporated herein by reference.

### TECHNICAL FIELD

The invention is in the field of therapeutics and medicinal chemistry. In particular, the invention concerns methods of treatment for cancer and inflammatory diseases that include administration of certain quinoxaline compounds.

### BACKGROUND ART

Cell signaling via 3′-phosphorylated phosphoinositides has been implicated in a variety of cellular processes, e.g., malignant transformation, growth factor signaling, inflammation, and immunity. The enzyme responsible for generating these phosphorylated signaling products, phosphatidylinositol 3-kinase (PI 3-kinase; PI3K), was originally identified as an activity associated with viral oncoproteins and growth factor receptor tyrosine kinases that phosphorylates phosphatidylinositol (PI) and its phosphorylated derivatives at the 3′-hydroxyl of the inositol ring.

The initial purification and molecular cloning of PI 3-kinase revealed that it was a heterodimer consisting of p85 and p110 subunits. Four distinct Class I PI3Ks have been identified, designated PI3K α, β, γ, and δ, each consisting of a distinct 110 kDa catalytic subunit and a regulatory subunit. More specifically, three of the catalytic subunits, i.e., p110α, p110β and p110δ, each interact with the same regulatory subunit, p85; whereas p110γ interacts with a distinct regulatory subunit, p101. The patterns of expression of each of these PI3Ks in human cells and tissues are also distinct.

Identification of the p110δ isoform of PI 3-kinase is described in Chantry, et al., J Biol Chem (1997) 272:19236-19241. It was observed that the human p110δ isoform is expressed in a tissue-restricted fashion. It is expressed at high levels in lymphocytes and lymphoid tissues, suggesting that the protein might play a role in PI 3-kinase-mediated signaling in the immune system.

PI 3-kinase activation, is believed to be involved in a range of cellular responses including cell growth, differentiation, and apoptosis.

The nonselective phosphoinositide 3-kinase (PI3K) inhibitors, LY294002 and wortmannin, have been shown to enhance destruction of tumor vasculature in irradiated endothelial cells (Edwards, et al., Cancer Res (2002) 62: 4671-4677). LY294002 and wortmannin do not distinguish among the four members of class I PI3Ks. For example, the IC50 values of wortmannin against each of the various class I PI3Ks are in the range of 1-10 nM. Similarly, the IC50 values for LY294002 against each of these PI3Ks is about 1 μM (Fruman, et al., Ann. Rev. Biochem (1998) 67:481-507). These inhibitors are not only nonselective with respect to class I PI3Ks, but are also potent inhibitors of DNA dependent protein kinase, FRAP-mTOR, smooth muscle myosin light chain kinase, and casein kinase 2 (Hartley, et al., Cell (1995) 82:849; Davies, et al., Biochem. J. (2000) 351:95; Brunn, et al., EMBO J. (1996) 15:5256).

Because p110α p110β, p110γ, and p110δ are expressed differentially by a wide variety of cell types, the administration of nonselective PI3K inhibitors such as LY294002 and wortmannin almost certainly will also affect cell types that may not be targeted for treatment. Therefore, the effective therapeutic dose of such nonselective inhibitors may be expected to exhibit non-selective biological effects, because otherwise non-targeted cell types will likely be affected, especially when such nonselective inhibitors are combined with cytotoxic therapies including but not limited to chemotherapy, radiation therapy, photodynamic therapies, radiofrequency ablation, and/or anti-angiogenic therapies.

There remains a need for safer and more effective methods of treating and preventing indications involving inflammatory conditions, autoimmune conditions and angiogenesis. Compounds that inhibit certain isoforms of PI3K have been shown to treat such disorders. Therefore, there remains a need for novel compounds that act as PI3K inhibitors, preferably with isoform selectivity patterns that are suited to treating these disorders with minimal off-target effects. The present invention provides such compounds as further described below.

### SUMMARY

The invention provides novel quinoxaline containing compounds and methods to treat cancer and inflammatory diseases with said compounds. In one aspect, the invention provides a compound of Formula I or a pharmaceutically acceptable salt thereof,

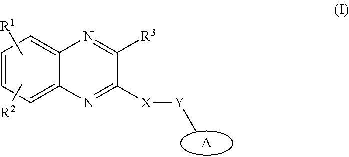

wherein A is a monocyclic or bicyclic ring system containing at least two nitrogen atoms, and at least one ring of the system is aromatic;

wherein A is optionally substituted with 1-3 substituents;

X is selected from the group consisting of C(R^{b})₂, CH₂CHR^{b}, and CH═C(R^{b});

Y is selected from the group consisting of null, S, SO, SO₂, NR^{d}, O, C(═O), OC(═O), C(═O)O, and NHC(═O)CH₂S;

R¹ and R², independently, are selected from the group consisting of hydrogen, halo, NO₂, CF₃, OCF₃, and CN, or from the group consisting of C₁₋₆alkyl, aryl, heteroaryl, NHC(═O)C₁₋₃alkyleneN(R^{a})₂, OR^{a}, N(R^{a})₂, OC(═O)R^{a}, C(═O)R^{a}, C(═O)OR^{a}, arylOR^{a}, Het, NR^{a}C(═O)C₁₋₃alkyleneC(═O)OR^{a}, arylOC₁₋₃alkyleneN(R^{a})₂, arylOC(═O)R^{a}, C₁₋₄alkyleneC(═O)OR^{a}, OC₁₋₄alkyleneC(═O)OR^{a}, C₁₋₄alkyleneOC₁₋₄alkyleneC(═O)OR^{a}, C(═O)NR^{a}SO₂R^{a}, C₁₋₄alkyleneN(R^{a})₂, C₂₋₆alkenyleneN(R^{a})₂, C(═O)NR^{a}C₁₋₄alkyleneOR^{a}, C(═O)NR^{a}C₁₋₄alkyleneHet, OC₂₋₄alkyleneN(R^{a})₂, OC₁₋₄alkyleneCH(OR^{a})CH₂N(R^{a})₂, OC₁₋₄alkyleneHet, OC₂₋₄alkyleneOR^{a}, OC₂₋₄alkyleneNR^{a}C(═O)OR^{a}, NR^{a}C₁₋₄alkyleneN(R^{a})₂, NR^{a}C(═O)R^{a}, NR^{a}C(═O)N(R^{a})₂, N(SO₂C₁₋₄alkyl)₂, NR^{a}(SO₂C₁₋₄alkyl), SO₂N(R^{a})₂, OSO₂CF₃, C₁₋₃alkylenearyl, C₁₋₄alkyleneHet, C₁₋₆alkyleneOR^{a}, C₁₋₃alkyleneN(R^{a})₂, C(═O)N(R^{a})₂, NHC(═O)C₁₋₃alkylenearyl, C₃₋₈cycloalkyl, C₃₋₈heterocycloalkyl, arylOC₁₋₃alkyleneN(R^{a})₂, arylOC(═O)R^{a}, NHC(═O)C₁₋₃alkyleneC₃₋₈heterocycloalkyl, NHC(═O)C₁₋₃alkyleneHet, OC₁₋₄alkyleneOC₁₋₄alkyleneC(═O)OR^{a}, C(═O)C₁₋₄alkyleneHet, and NHC(═O)haloC₁₋₆alkyl, each of which is optionally substituted;

or R¹ and R² are taken together to form a 3- or 4-membered alkylene or alkenylene chain component of a 5- or 6-membered ring, optionally containing at least one heteroatom selected from the group consisting of N, O, and S;

R³ is hydrogen or is a member selected from the group consisting of C₁₋₆alkyl, C₃₋₈cycloalkyl, C₃₋₈heterocycloalkyl, C₁₋₄alkylenecycloalkyl, C₂₋₆alkenyl, C₁₋₃alkylenearyl, arylC₁₋₃alkyl, C(═O)R^{a}, aryl, heteroaryl, C(═O)OR^{a}, C(═O)N(R^{a})₂, C(═S)N(R^{a})₂, SO₂R^{a}, SO₂N(R^{a})₂, S(═O)R^{a}, S(═O)N(R^{a})₂, C(═O)NR^{a}C₁₋₄alkyleneOR^{a}, C(═O)NR^{a}C₁₋₄alkyleneHet, C(═O)C₁₋₄alkylenearyl, C(═O)C₁₋₄alkyleneheteroaryl, and C₁₋₄alkylenearyl, each of which is optionally substituted with 1-3 substituents;

each R^{a} is independently selected from hydrogen or from the group consisting of C₁₋₆alkyl, C₃₋₈cycloalkyl, C₃₋₈heterocycloalkyl, C₁₋₃alkyleneN(R^{c})₂, aryl, arylC₁₋₃alkyl, C₁₋₃alkylenearyl, heteroaryl, heteroarylC₁₋₃alkyl, and C₁₋₃alkyleneheteroaryl, each of which is optionally substituted;

or two R^{a} groups on the same atom or on adjacent atoms are taken together to form a 5- or 6-membered ring, optionally containing at least one heteroatom;

each R^{b} is independently selected from the group consisting of hydrogen, halo and CN or from the group consisting of C₁₋₆alkyl, C₁₋₆haloalkyl, C(═O)R^{a}, C(═O)OR^{a}, heteroC₁₋₃alkyl, C₁₋₃alkyleneheteroC₁₋₃alkyl, arylheteroC₁₋₃alkyl, aryl, heteroaryl, arylC₁₋₃alkyl, heteroarylC₁₋₃alkyl, C₁₋₃alkylenearyl, and C₁₋₃alkyleneheteroaryl, each of which is optionally substituted; or R^{b} and R^{d} can be taken together to form a 5-7 membered optionally substituted ring;

each R^{c} is independently selected from hydrogen or from the group consisting of C₁₋₆alkyl, C₃₋₈cycloalkyl, aryl, and heteroaryl, each of which is optionally substituted;

wherein R^{d} is H or C₁₋₁₀acyl; or R^{d} and R^{b}, if X comprises R^{b}, can be taken together to form a 5-7 membered optionally substituted ring; and

each Het is a 5- or 6-membered heterocyclic ring, wherein said heterocyclic ring is saturated, partially unsaturated or aromatic, and said heterocyclic ring contains at least one heteroatom selected from the group consisting of N, O, and S; wherein Het is optionally substituted with 1-3 substituents.

In particular embodiments, the acyclic linker between the purinyl ring and the quinoxaline ring comprises a chiral center. In some embodiments, the chiral center is the S-enantiomer.

In another aspect, the invention provides a method to prevent or treat a condition in a subject in need thereof, wherein said condition is an inflammatory condition or cancer, comprising administering to the subject a therapeutically effective amount of a compound described herein.

In yet another aspect, the invention provides for a pharmaceutical composition comprising any compound described herein; and at least one pharmaceutically acceptable excipient.

### MODES OF CARRYING OUT THE INVENTION

The invention provides novel quinoxaline containing compounds and methods to treat cancer and inflammatory diseases with said compounds. In some methods of the invention wherein a selective PI3K inhibitor is employed, it is preferred that the compound be at least 10-fold selective for inhibition of at least one particular PI3K isoform relative to one or more other Type I PI3K isoforms in a cell-based assay. In some embodiments, the compound is at least 20-fold selective for at least one particular isoform PI3K isoform relative to one or more other Type I PI3K isoforms in a cell-based assay. In other embodiments, the compound is at least 50-fold selective for inhibition of at least one PI3K isoform relative to one or more other Type I PI3K isoforms in a cell-based assay. In particular embodiments, the invention provides compounds that are selective for PI3Kδ relative to at least one of PI3Kα and PI3Kβ. In other embodiments, the invention provides compounds that are selective inhibitors of PI3Kδ and γ relative to at least one of PI3Kα and PI3Kβ.

In one aspect, the invention provides a compound of Formula I or a pharmaceutically acceptable salt thereof,

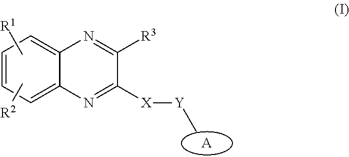

wherein A is a monocyclic or bicyclic ring system containing at least two nitrogen atoms, and at least one ring of the system is aromatic;

wherein A is optionally substituted with 1-3 substituents;

X is selected from the group consisting of C(R^{b})₂, CH₂CHR^{b}, and CH═C(R^{b});

Y is selected from the group consisting of null, S, SO, SO₂, NR^{d}, O, C(═O), OC(═O), C(═O)O, and NHC(═O)CH₂S;

R¹ and R², independently, are selected from the group consisting of hydrogen, halo, NO₂, CF₃, OCF₃, and CN, or from the group consisting of C₁₋₆alkyl, aryl, heteroaryl, NHC(═O)C₁₋₃alkyleneN(R^{a})₂, OR^{a}, N(R^{a})₂, OC(═O)R^{a}, C(═O)R^{a}, C(═O)OR^{a}, arylOR^{a}, Het, NR^{a}C(═O)C₁₋₃alkyleneC(═O)OR^{a}, arylOC₁₋₃alkyleneN(R^{a})₂, arylOC(═O)R^{a}, C₁₋₄alkyleneC(═O)OR^{a}, OC₁₋₄alkyleneC(═O)OR^{a}, C₁₋₄alkyleneOC₁₋₄alkyleneC(═O)OR^{a}, C(═O)NR^{a}SO₂R^{a}, C₁₋₄alkyleneN(R^{a})₂, C₂₋₆alkenyleneN(R^{a})₂, C(═O)NR^{a}C₁₋₄alkyleneOR^{a}, C(═O)NR^{a}C₁₋₄alkyleneHet, OC₂₋₄alkyleneN(R^{a})₂, OC₁₋₄alkyleneCH(OR^{a})CH₂N(R^{a})₂, OC₁₋₄alkyleneHet, OC₂₋₄alkyleneOR^{a}, OC₂₋₄alkyleneNR^{a}C(═O)OR^{a}, NR^{a}C₁₋₄alkyleneN(R^{a})₂, NR^{a}C(═O)R^{a}, NR^{a}C(═O)N(R^{a})₂, N(SO₂C₁₋₄alkyl)₂, NR^{a}(SO₂C₁₋₄alkyl), SO₂N(R^{a})₂, OSO₂CF₃, C₁₋₃alkylenearyl, C₁₋₄alkyleneHet, C₁₋₆alkyleneOR^{a}, C₁₋₃alkyleneN(R^{a})₂, C(═O)N(R^{a})₂, NHC(═O)C₁₋₃alkylenearyl, C₃₋₈cycloalkyl, C₃₋₈heterocycloalkyl, arylOC₁₋₃alkyleneN(R^{a})₂, arylOC(═O)R^{a}, NHC(═O)C₁₋₃alkyleneC₃₋₈heterocycloalkyl, NHC(═O)C₁₋₃alkyleneHet, OC₁₋₄alkyleneOC₁₋₄alkyleneC(═O)OR^{a}, C(═O)C₁₋₄alkyleneHet, and NHC(═O)haloC₁₋₆alkyl, each of which is optionally substituted;

or R¹ and R² are taken together to form a 3- or 4-membered alkylene or alkenylene chain component of a 5- or 6-membered ring, optionally containing at least one heteroatom selected from the group consisting of N, O, and S;

R³ is hydrogen or is a member selected from the group consisting of C₁₋₆alkyl, C₃₋₈cycloalkyl, C₃₋₈heterocycloalkyl, C₁₋₄alkylenecycloalkyl, C₂₋₆alkenyl, C₁₋₃alkylenearyl, arylC₁₋₃alkyl, C(═O)R^{a}, aryl, heteroaryl, C(═O)OR^{a}, C(═O)N(R^{a})₂, C(═S)N(R^{a})₂, SO₂R^{a}, SO₂N(R^{a})₂, S(═O)R^{a}, S(═O)N(R^{a})₂, C(═O)NR^{a}C₁₋₄alkyleneOR^{a}, C(═O)NR^{a}C₁₋₄alkyleneHet, C(═O)C₁₋₄alkylenearyl, C(═O)C₁₋₄alkyleneheteroaryl, and C₁₋₄alkylenearyl, each of which is optionally substituted with 1-3 substituents;

each R^{a} is independently selected from hydrogen or from the group consisting of C₁₋₆alkyl, C₃₋₈cycloalkyl, C₃₋₈heterocycloalkyl, C₁₋₃alkyleneN(R^{c})₂, aryl, arylC₁₋₃alkyl, C₁₋₃alkylenearyl, heteroaryl, heteroarylC₁₋₃alkyl, and C₁₋₃alkyleneheteroaryl, each of which is optionally substituted;

or two R^{a} groups on the same atom or on adjacent atoms are taken together to form a 5- or 6-membered ring, optionally containing at least one heteroatom;

each R^{b} is independently selected from the group consisting of hydrogen, halo and CN or from the group consisting of C₁₋₆alkyl, C₁₋₆haloalkyl, C(═O)R^{a}, C(═O)OR^{a}, heteroC₁₋₃alkyl, C₁₋₃alkyleneheteroC₁₋₃alkyl, arylheteroC₁₋₃alkyl, aryl, heteroaryl, arylC₁₋₃alkyl, heteroarylC₁₋₃alkyl, C₁₋₃alkylenearyl, and C₁₋₃alkyleneheteroaryl, each of which is optionally substituted; or R^{b} and R^{d} can be taken together to form a 5-7 membered optionally substituted ring;

each R^{c} is independently selected from hydrogen or from the group consisting of C₁₋₆alkyl, C₃₋₈cycloalkyl, aryl, and heteroaryl, each of which is optionally substituted;

wherein R^{d} is H or C₁₋₁₀acyl; or R^{d} and R^{b}, if X comprises R^{b}, can be taken together to form a 5-7 membered optionally substituted ring; and

each Het is a 5- or 6-membered heterocyclic ring, wherein said heterocyclic ring is saturated, partially unsaturated or aromatic, and said heterocyclic ring contains at least one heteroatom selected from the group consisting of N, O, and S; wherein Het is optionally substituted with 1-3 substituents.

In some embodiments, the quinoxaline group is substituted with a substituent at position 5. In some embodiments, the quinoxaline group is substituted with a substituent at position 6. In some embodiments, the quinoxaline group is substituted with a substituent at position 7. In some embodiments, the quinoxaline group is substituted with a substituent at position 8. The enumerated positions of the quinoxaline group is shown here:

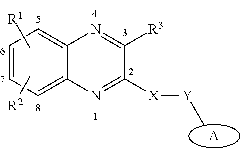

“Optionally substituted” as used herein indicates that the particular group or groups being described may have no non-hydrogen substituents (i.e., it can be unsubstituted), or the group or groups may have one or more non-hydrogen substituents. If not otherwise specified, the total number of such substituents that may be present is equal to the number of H atoms present on the unsubstituted form of the group being described. Typically, a group will contain up to three (0-3) substituents.

In some embodiments, compound I comprises a chiral center in the linker between the two ring systems, represented as —X—Y— in Formula I. In such embodiments, the compound is sometimes preferably optically active, meaning it consists of predominantly one of two enantiomers. In some embodiments, the compound is used in an optically active form, which contains predominantly the S-enantiomer at this chiral center. Such compounds may be synthesized in optically active form, or they may be prepared in racemic form (containing equal amounts of R and S isomers), and then the isomers may be separated.

While it is sometimes preferable to substantially exclude the enantiomeric R isomer from the compound of Formula (I) when the compound comprises a chiral center in this linker, the compounds and methods of the invention can be practiced with mixtures of R and S isomers as well. In certain embodiments, the compound is preferably used as a non-racemic mixture wherein the S isomer is the major component of the mixture. Typically such mixture will contain no more than about 10% of the R isomer, meaning the ratio of S to R isomers is at least about 9:1, and preferably less than 5% of the R-isomer, meaning the ratio of S to R enantiomers is at least about 19:1. In some embodiments the compound used has less than 2% R enantiomer, meaning it has an enantiomeric excess of at least about 96%. In some embodiments, the compound has an enantiomeric excess of at least 98%. In some embodiments, the compound has an enantiomeric excess of at least 99%.

In some embodiments, the compositions and methods of the invention utilize an optically active form of Compound I (the compound of Formula I) when it comprises a chiral center in the linker between the two ring systems, meaning in each instance, the compound is optically active and contains predominantly the S-enantiomer, although it may contain the R-enantiomer of Compound I as a minor component. For clarity, where a dosage of a compound of Formula I, or a dosage of Compound I is described herein, the dosage refers to the weight of the compound of Formula I, including each enantiomer that is present. Thus, a dosage of 100 mg of Compound I as used herein, for example, refers to the weight of the mixture of enantiomers rather than the weight of the S-enantiomer specifically. It could, for example, refer to 100 mg of a 9:1 mixture of S and R enantiomers, which would contain about 90 mg of the S enantiomer, or to 100 mg of a 19:1 mixture of S and R enantiomers, which would contain about 95 mg of the S enantiomer.

The invention also included compounds of Formula I in which from 1 to n hydrogens attached to a carbon atom is/are replaced by deuterium, in which n is the number of hydrogens in the molecule. Such compounds exhibit increased resistance to metabolism, and are thus useful for increasing the half life of any compound of Formula I when administered to a mammal. See, for example, Foster, “Deuterium Isotope Effects in Studies of Drug Metabolism”, Trends Pharmacol. Sci. 5(12):524-527 (1984). Such compounds are synthesized by means well known in the art, for example by employing starting materials in which one or more hydrogens have been replaced by deuterium.

In some embodiments, X is C(R^{b})₂ or CH₂CHR^{b}; and wherein X has a chiral center. In some embodiments, the chiral center is the S-enantiomer. In some embodiments, X is C(R^{b})₂ or X is CHR^{b}. In specific embodiments, X is selected from the group consisting of CH₂, CH(CH₂)₀₋₂CH₃, CHCH(CH₃)₂, C(CH₃)₂, and CHCH((CH₂)₀₋₁CH₃)₂, each of which is optionally substituted. In other specific embodiments, X is selected from the group consisting of CH₂, CHCH₃, and CHCH₂CH₃.

In some embodiments A is selected from the group consisting of

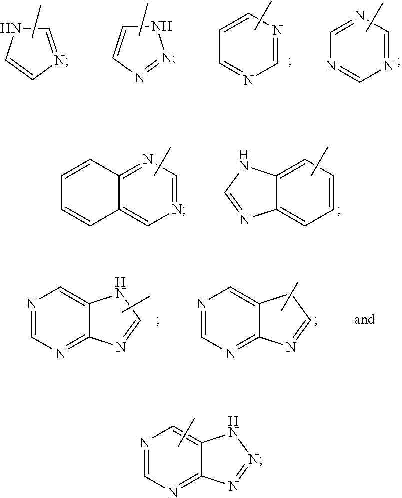

each of which is optionally substituted, including stable tautomers of these structures. In these structures, A can be connected to Y in Formula I at any position of A that is available for substitution. In specific embodiments, A is a purinyl ring. In particular embodiments, X or Y connects to the purinyl ring at position 6 or 9 of the purinyl ring. In particular embodiments, X or Y connects to the purinyl ring at position 6 of the purinyl ring. In particular embodiments, X or Y connects to the purinyl ring at position 9 of the purinyl ring. The purine ring structure and numbering is shown here:

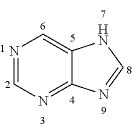

In some embodiments, A is optionally substituted with 1-3 substituents independently selected from the group consisting of hydrogen, halo, NO₂, CF₃, OCF₃, and CN, or from the group consisting of C₁₋₆alkyl, aryl, heteroaryl, NHC(═O)C₁₋₃alkyleneN(R^{a})₂, OR^{a}, N(R^{a})₂, OC(═O)R^{a}, C(═O)R^{a}, C(═O)OR^{a}, arylOR^{a}, Het, NR^{a}C(═O)C₁₋₃alkyleneC(═O)OR^{a}, arylOC₁₋₃alkyleneN(R^{a})₂, arylOC(═O)R^{a}, C₁₋₄alkyleneC(═O)OR^{a}, OC₁₋₄alkyleneC(═O)OR^{a}, C₁₋₄alkyleneOC₁₋₄alkyleneC(═O)OR^{a}, C(═O)NR^{a}SO₂R^{a}, C₁₋₄alkyleneN(R^{a})₂, C₂₋₆alkenyleneN(R^{a})₂, C(═O)NR^{a}C₁₋₄alkyleneOR^{a}, C(═O)NR^{a}C₁₋₄alkyleneHet, OC₂₋₄alkyleneN(R^{a})₂, OC₁₋₄alkyleneCH(OR^{a})CH₂N(R^{a})₂, OC₁₋₄alkyleneHet, OC₂₋₄alkyleneOR^{a}, OC₂₋₄alkyleneNR^{a}C(═O)OR^{a}, NR^{a}C₁₋₄alkyleneN(R^{a})₂, NR^{a}C(═O)R^{a}, NR^{a}C(═O)N(R^{a})₂, N(SO₂C₁₋₄alkyl)₂, NR^{a}(SO₂C₁₋₄alkyl), SO₂N(R^{a})₂, OSO₂CF₃, C₁₋₃alkylenearyl, C₁₋₄alkyleneHet, C₁₋₆alkyleneOR^{a}, C₁₋₃alkyleneN(R^{a})₂, C(═O)N(R^{a})₂, NHC(═O)C₁₋₃alkylenearyl, C₃₋₈cycloalkyl, C₃₋₈heterocycloalkyl, arylOC₁₋₃alkyleneN(R^{a})₂, arylOC(═O)R^{a}, NHC(═O)C₁₋₃alkyleneC₃₋₈heterocycloalkyl, NHC(═O)C₁₋₃alkyleneHet, OC₁₋₄alkyleneOC₁₋₄alkyleneC(═O)OR^{a}, C(═O)C₁₋₄alkyleneHet, and NHC(═O)haloC₁₋₆alkyl, each of which is optionally substituted. In specific embodiments, A is optionally substituted with 1-3 substituents independently selected from the group consisting of N(R^{a})₂, halo, CN, C1-6alkyl, C1-6haloalkyl C(═O)R^{a}, and C(═O)OR^{a}. In other embodiments, A is optionally substituted with 1-3 substituents independently selected from the group consisting of hydrogen, F, Cl, Br, NO₂, NH₂, CN, CF₃, and OCF₃, or from the group consisting of methyl, ethyl, propyl, butyl, phenyl, heteroaryl, OR^{a}, N(R^{a})₂, OC(═O)R^{a}, C(═O)R^{a}, C(═O)OR^{a}, Het, each of which is optionally substituted. In more specific embodiments, the purinyl ring is optionally substituted at positions 2 or 6. In more specific embodiments, the purinyl ring is optionally substituted at position 2. In alternative embodiments, the purinyl ring is optionally substituted at position 6. In alternative embodiments, the purinyl ring is optionally substituted at position 8.

In some embodiments, wherein gamma potency is desired, group A of the quinoxaline compound comprises an amino-substituted purinyl ring. Examples of increased gamma potency related to aminopurinyl rings include compound Q17 and Q15. Compared to analogs that do not contain the amino substituent, Q16 and Q14, respectively, compounds having amino substituted on the purine ring have a 30× and 8× increase in gamma potency, respectively.

In specific embodiments, A is optionally substituted with NH₂. In more specific embodiments, A is a purinyl ring substituted with NH₂. In further specific embodiments, A is a purinyl ring substituted with NH₂ at position 2 of the purinyl ring. In other embodiments, A is a purinyl ring substituted with NH₂ at position 6 of the purinyl ring.

R³ in some embodiments can be an optionally substituted C₁₋₆alkyl, C₃₋₈cycloalkyl, C₃₋₈heterocycloalkyl, C₁₋₄alkylenecycloalkyl, C₂₋₆alkenyl, C₁₋₃alkylenearyl, arylC₁₋₃alkyl, C(═O)R^{a}, aryl, or heteroaryl. In certain embodiments, R³ is optionally substituted aryl, and in specific embodiments, R³ is substituted or unsubstituted phenyl. Suitably substituted phenyls include mono-, di-, and tri-substituted phenyls, having at least one substituent ortho to the position of R³ that is linked to N in Formula I; or having at least one substituent meta to the position of R³ that is linked to N in Formula I; or having at least one substituent para to the position of R³ that is linked to N in Formula I.

In some embodiments, R³ is optionally substituted with 1-3 substituents independently selected from the group consisting of halo, NO₂, CF₃, OCF₃, and CN, or from the group consisting of C₁₋₆alkyl, aryl, heteroaryl, NHC(═O)C₁₋₃alkyleneN(R^{a})₂, OR^{a}, N(R^{a})₂, OC(═O)R^{a}, C(═O)R^{a}, C(═O)OR^{a}, arylOR^{a}, Het, NR^{a}C(═O)C₁₋₃alkyleneC(═O)OR^{a}, arylOC₁₋₃alkyleneN(R^{a})₂, arylOC(═O)R^{a}, C₁₋₄alkyleneC(═O)OR^{a}, OC₁₋₄alkyleneC(═O)OR^{a}, C₁₋₄alkyleneOC₁₋₄alkyleneC(═O)OR^{a}, C(═O)NR^{a}SO₂R^{a}, C₁₋₄alkyleneN(R^{a})₂, C₂₋₆alkenyleneN(R^{a})₂, C(═O)NR^{a}C₁₋₄alkyleneOR^{a}, C(═O)NR^{a}C₁₋₄alkyleneHet, OC₂₋₄alkyleneN(R^{a})₂, OC₁₋₄alkyleneCH(OR^{a})CH₂N(R^{a})₂, OC₁₋₄alkyleneHet, OC₂₋₄alkyleneOR^{a}, OC₂₋₄alkyleneNR^{a}C(═O)OR^{a}, NR^{a}C₁₋₄alkyleneN(R^{a})₂, NR^{a}C(═O)R^{a}, NR^{a}C(═O)N(R^{a})₂, N(SO₂C₁₋₄alkyl)₂, NR^{a}(SO₂C₁₋₄alkyl), SO₂N(R^{a})₂, OSO₂CF₃, C₁₋₃alkylenearyl, C₁₋₄alkyleneHet, C₁₋₆alkyleneOR^{a}, C₁₋₃alkyleneN(R^{a})₂, C(═O)N(R^{a})₂, NHC(═O)C₁₋₃alkylenearyl, C₃₋₈cycloalkyl, C₃₋₈heterocycloalkyl, arylOC₁₋₃alkyleneN(R^{a})₂, arylOC(═O)R^{a}, NHC(═O)C₁₋₃alkyleneC₃₋₈heterocycloalkyl, NHC(═O)C₁₋₃alkyleneHet, OC₁₋₄alkyleneOC₁₋₄alkyleneC(═O)OR^{a}, C(═O)C₁₋₄alkyleneHet, and NHC(═O)haloC₁₋₆alkyl, each of which is optionally substituted; or two substituents are taken together to form a 3- or 4-membered alkylene or alkenylene chain component of a 5- or 6-membered ring, optionally containing at least one heteroatom selected from the group consisting of N, O, and S.

In more specific embodiments, R³ is optionally substituted with 1-3 substituents independently selected from the group consisting of hydrogen, F, Cl, Br, NO₂, CN, CF₃, and OCF₃, or from the group consisting of methyl, ethyl, propyl, butyl, phenyl, heteroaryl, OR^{a}, N(R^{a})₂, OC(═O)R^{a}, C(═O)R^{a}, C(═O)OR^{a}, Het, each of which is optionally substituted.

In some embodiments, R³ is optionally substituted aryl. In some embodiments, R³ is phenyl optionally substituted with 1-3 substituents independently selected from the group consisting of N(R^{a})₂, halo, CN, C₁₋₆alkyl, OR^{a}, C₁₋₆haloalkyl C(═O)R^{a}, and C(═O)OR^{a}. In specific embodiments R³ is phenyl optionally substituted with 1-3 substituents independently selected from the group consisting of F, Br, Cl, NH₂, CN, CF₃, OCF₃ and NO₂, or the group consisting of methyl, ethyl, propyl, isopropyl, butyl, and tert-butyl, each of which is further optionally substituted. Specific substituents suitable for R³ include F, Cl, Me, CF₃, and CN.

In some embodiments, R¹ and R², independently, are selected from the group consisting of hydrogen, F, Cl, Br, NO₂, CF₃, OCF₃, and CN, or from the group consisting of methyl, ethyl, propyl, butyl, phenyl, heteroaryl, OR^{a}, N(R^{a})₂, OC(═O)R^{a}, C(═O)R^{a}, C(═O)OR^{a}, each of which is optionally substituted. In certain embodiments each R¹ and R² is selected from H, Cl, F, Me, and Br, and in some embodiments, at least one of R¹ and R² is H.

In some embodiments, each R^{b} is selected from the group consisting of hydrogen, halo, and CN or from the group consisting of methyl, ethyl, propyl, butyl, C(═O)R^{a}, and C(═O)OR^{a}, each of which may be optionally substituted. Preferred groups for R^{b} include H, Me, and Et.

In some embodiments, Y is NH, and in other embodiments Y is S.

In some embodiments, the compound of Formula I is represented by the Formula II

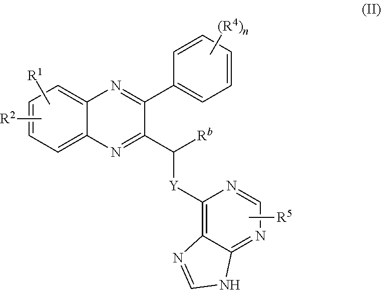

wherein each R⁴ is independently selected from the group consisting of hydrogen, halo, NO₂, CF₃, OCF₃, and CN, or from the group consisting of C₁₋₆alkyl, aryl, heteroaryl, NHC(═O)C₁₋₃alkyleneN(R^{a})₂, OR^{a}, N(R^{a})₂, OC(═O)R^{a}, C(═O)R^{a}, C(═O)OR^{a}, arylOR^{a}, Het, NR^{a}C(═O)C₁₋₃alkyleneC(═O)OR^{a}, arylOC₁₋₃alkyleneN(R^{a})₂, arylOC(═O)R^{a}, C₁₋₄alkyleneC(═O)OR^{a}, OC₁₋₄alkyleneC(═O)OR^{a}, C₁₋₄alkyleneOC₁₋₄alkyleneC(═O)OR^{a}, C(═O)NR^{a}SO₂R^{a}, C₁₋₄alkyleneN(R^{a})₂, C₂₋₆alkenyleneN(R^{a})₂, C(═O)NR^{a}C₁₋₄alkyleneOR^{a}, C(═O)NR^{a}C₁₋₄alkyleneHet, OC₂₋₄alkyleneN(R^{a})₂, OC₁₋₄alkyleneCH(OR^{a})CH₂N(R^{a})₂, OC₁₋₄alkyleneHet, OC₂₋₄alkyleneOR^{a}, OC₂₋₄alkyleneNR^{a}C(═O)OR^{a}, NR^{a}C₁₋₄alkyleneN(R^{a})₂, NR^{a}C(═O)R^{a}, NR^{a}C(═O)N(R^{a})₂, N(SO₂C₁₋₄alkyl)₂, NR^{a}(SO₂C₁₋₄alkyl), SO₂N(R^{a})₂, OSO₂CF₃, C₁₋₃alkylenearyl, C₁₋₄alkyleneHet, C₁₋₆alkyleneOR^{a}, C₁₋₃alkyleneN(R^{a})₂, C(═O)N(R^{a})₂, NHC(═O)C₁₋₃alkylenearyl, C₃₋₈cycloalkyl, C₃₋₈heterocycloalkyl, arylOC₁₋₃alkyleneN(R^{a})₂, arylOC(═O)R^{a}, NHC(═O)C₁₋₃alkyleneC₃₋₈heterocycloalkyl, NHC(═O)C₁₋₃alkyleneHet, OC₁₋₄alkyleneOC₁₋₄alkyleneC(═O)OR^{a}, C(═O)C₁₋₄alkyleneHet, and NHC(═O)haloC₁₋₆alkyl, each of which is optionally substituted;

or two R⁴ groups are taken together to form a 3- or 4-membered alkylene or alkenylene chain component of a 5- or 6-membered ring, optionally containing at least one heteroatom selected from the group consisting of N, O, and S;

n is 0-3; and

R⁵ is selected from the group consisting of hydrogen, halo, NO₂, CF₃, OCF₃, and CN, or from the group consisting of C₁₋₆alkyl, aryl, heteroaryl, NHC(═O)C₁₋₃alkyleneN(R^{a})₂, OR^{a}, N(R^{a})₂, OC(═O)R^{a}, C(═O)R^{a}, C(═O)OR^{a}, arylOR^{a}, Het, NR^{a}C(═O)C₁₋₃alkyleneC(═O)OR^{a}, arylOC₁₋₃alkyleneN(R^{a})₂, arylOC(═O)R^{a}, C₁₋₄alkyleneC(═O)OR^{a}, OC₁₋₄alkyleneC(═O)OR^{a}, C₁₋₄alkyleneOC₁₋₄alkyleneC(═O)OR^{a}, C(═O)NR^{a}SO₂R^{a}, C₁₋₄alkyleneN(R^{a})₂, C₂₋₆alkerlylerleN(R^{a})₂, C(═O)NR^{a}C₁₋₄alkyleneOR^{a}, C(═O)NR^{a}C₁₋₄alkyleneHet, OC₂₋₄alkyleneN(R^{a})₂, OC₁₋₄alkyleneCH(OR^{a})CH₂N(R^{a})₂, OC₁₋₄alkyleneHet, OC₂₋₄alkyleneOR^{a}, OC₂₋₄alkyleneNR^{a}C(═O)OR^{a}, NR^{a}C₁₋₄alkyleneN(R^{a})₂, NR^{a}C(═O)R^{a}, NR^{a}C(═O)N(R^{a})₂, N(SO₂C₁₋₄alkyl)₂, NR^{a}(SO₂C₁₋₄alkyl), SO₂N(R^{a})₂, OSO₂CF₃, C₁₋₃alkylenearyl, C₁₋₄alkyleneHet, C₁₋₆alkyleneOR^{a}, C₁₋₃alkyleneN(R^{a})₂, C(═O)N(R^{a})₂, NHC(═O)C₁₋₃alkylenearyl, C₃₋₈cycloalkyl, C₃₋₈heterocycloalkyl, arylOC₁₋₃alkyleneN(R^{a})₂, arylOC(═O)R^{a}, NHC(═O)C₁₋₃alkyleneC₃₋₈heterocycloalkyl, NHC(═O)C₁₋₃alkyleneHet, OC₁₋₄alkyleneOC₁₋₄alkyleneC(═O)OR^{a}, C(═O)C₁₋₄alkyleneHet, and NHC(═O)haloC₁₋₆alkyl, each of which is optionally substituted.

In specific embodiments, each R⁴ is independently selected from the group consisting of hydrogen, F, Cl, Br, NO₂, CN, CF₃, and OCF₃, or from the group consisting of methyl, ethyl, propyl, butyl, phenyl, heteroaryl, OR^{a}, N(R^{a})₂, OC(═O)R^{a}, C(═O)R^{a}, C(═O)OR^{a}, Het, each of which is optionally substituted.

In specific embodiments, R⁵ is selected from the group consisting of hydrogen, F, Cl, Br, NH₂, NO₂, CN, CF₃, and OCF₃, or from the group consisting of methyl, ethyl, propyl, butyl, phenyl, heteroaryl, OR^{a}, N(R^{a})₂, OC(═O)R^{a}, C(═O)R^{a}, C(═O)OR^{a}, Het, each of which is optionally substituted. H, Me, CF₃, F, or NH₂ is sometimes preferred, and in many embodiments R⁵ is H.

In some embodiments of Formula II, R¹ and R², independently, are selected from the group consisting of hydrogen, F, Cl, Br, NO₂, CF₃, OCF₃, and CN, or from the group consisting of methyl, ethyl, propyl, butyl, phenyl, heteroaryl, OR^{a}, N(R^{a})₂, OC(═O)R^{a}, C(═O)R^{a}, C(═O)OR^{a}, each of which is optionally substituted; H, F, Cl, Br, and Me are sometimes preferred.

In such embodiments of Formula II, R^{b} is selected from the group consisting of hydrogen, halo, and CN or from the group consisting of methyl, ethyl, propyl, butyl, C(═O)R^{a}, and C(═O)OR^{a}, each of which may be optionally substituted; H, Me, Ethyl, and propyl are sometimes preferred.

In the foregoing embodiments of Formula II, each R⁴ is independently selected from the group consisting of hydrogen, F, Cl, Br, NO₂, CN, CF₃, and OCF₃, or from the group consisting of methyl, ethyl, propyl, butyl, phenyl, heteroaryl, OR^{a}, N(R^{a})₂, OC(═O)R^{a}, C(═O)R^{a}, C(═O)OR^{a}, Het, each of which is optionally substituted; H, F, Cl, Br, CN, CF₃, and Me are sometimes preferred.

In such embodiments of Formula II, n is 0-2. In some embodiments n is 0; in other embodiments, n is 1; and in other embodiments, n is 2. Where n is 1 and R⁴ is not H, it is sometimes preferred for R⁴ to be positioned ortho to the point at which the phenyl ring on which R⁴ is located is attached to the N of the quinoxaline ring.

In such embodiments of Formula II, R⁵ is selected from the group consisting of hydrogen, F, Cl, Br, NO₂, CN, CF₃, and OCF₃, or from the group consisting of methyl, ethyl, propyl, butyl, phenyl, heteroaryl, OR^{a}, N(R^{a})₂, OC(═O)R^{a}, C(═O)R^{a}, C(═O)OR^{a}, Het, each of which is optionally substituted. In certain of these embodiments, R⁵ is H, F, Me, or NH₂.

In specific embodiments, R⁵ is NH₂. In further specific embodiments, R⁵ is NH₂ at position 2 of the purinyl ring. In further specific embodiments, R⁵ is NH₂ at position 6 of the purinyl ring. In further specific embodiments, R⁵ is NH₂ at position 8 of the purinyl ring.

In another aspect, the compound of Formula I is a compound of Formula III:

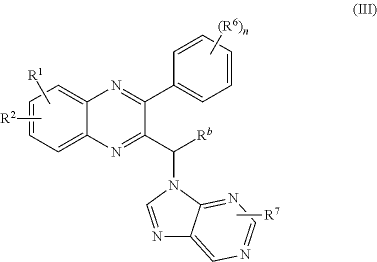

wherein R^{b} is selected from the group consisting of hydrogen, halo, and CN or from the group consisting of C₁₋₆alkyl, C(═O)R^{a}, and C(═O)OR^{a}, each of which may be optionally substituted;

each R⁶ is independently selected from the group consisting of hydrogen, halo, NO₂, CF₃, OCF₃, and CN, or from the group consisting of C₁₋₆alkyl, aryl, heteroaryl, NHC(═O)C₁₋₃alkyleneN(R^{a})₂, OR^{a}, N(R^{a})₂, OC(═O)R^{a}, C(═O)R^{a}, C(═O)OR^{a}, arylOR^{a}, Het, NR^{a}C(═O)C₁₋₃alkyleneC(═O)OR^{a}, arylOC₁₋₃alkyleneN(R^{a})₂, arylOC(═O)R^{a}, C₁₋₄alkyleneC(═O)OR^{a}, OC₁₋₄alkyleneC(═O)OR^{a}, C₁₋₄alkyleneOC₁₋₄alkyleneC(═O)OR^{a}, C(═O)NR^{a}SO₂R^{a}, C₁₋₄alkyleneN(R^{a})₂, C₂₋₆alkerlylerleN(R^{a})₂, C(═O)NR^{a}C₁₋₄alkyleneOR^{a}, C(═O)NR^{a}C₁₋₄alkyleneHet, OC₂₋₄alkyleneN(R^{a})₂, OC₁₋₄alkyleneCH(OR^{a})CH₂N(R^{a})₂, OC₁₋₄alkyleneHet, OC₂₋₄alkyleneOR^{a}, OC₂₋₄alkyleneNR^{a}C(═O)OR^{a}, NR^{a}C₁₋₄alkyleneN(R^{a})₂, NR^{a}C(═O)R^{a}, NR^{a}C(═O)N(R^{a})₂, N(SO₂C₁₋₄alkyl)₂, NR^{a}(SO₂C₁₋₄alkyl), SO₂N(R^{a})₂, OSO₂CF₃, C₁₋₃alkylenearyl, C₁₋₄alkyleneHet, C₁₋₆alkyleneOR^{a}, C₁₋₃alkyleneN(R^{a})₂, C(═O)N(R^{a})₂, NHC(═O)C₁₋₃alkylenearyl, C₃₋₈cycloalkyl, C₃₋₈heterocycloalkyl, arylOC₁₋₃alkyleneN(R^{a})₂, arylOC(═O)R^{a}, NHC(═O)C₁₋₃alkyleneC₃₋₈heterocycloalkyl, NHC(═O)C₁₋₃alkyleneHet, OC₁₋₄alkyleneOC₁₋₄alkyleneC(═O)OR^{a}, C(═O)C₁₋₄alkyleneHet, and NHC(═O)haloC₁₋₆alkyl, each of which is optionally substituted;

or two R⁶ groups are taken together to form a 3- or 4-membered alkylene or alkenylene chain component of a 5- or 6-membered ring, optionally containing at least one heteroatom selected from the group consisting of N, O and S;

n is 0-3; and

R⁷ is selected from the group consisting of hydrogen, halo, NO₂, CF₃, OCF₃, and CN, or from the group consisting of C₁₋₆alkyl, aryl, heteroaryl, NHC(═O)C₁₋₃alkyleneN(R^{a})₂, OR^{a}, N(R^{a})₂, OC(═O)R^{a}, C(═O)R^{a}, C(═O)OR^{a}, arylOR^{a}, Het, NR^{a}C(═O)C₁₋₃alkyleneC(═O)OR^{a}, arylOC₁₋₃alkyleneN(R^{a})₂, arylOC(═O)R^{a}, C₁₋₄alkyleneC(═O)OR^{a}, OC₁₋₄alkyleneC(═O)OR^{a}, C₁₋₄alkyleneOC₁₋₄alkyleneC(═O)OR^{a}, C(═O)NR^{a}SO₂R^{a}, C₁₋₄alkyleneN(R^{a})₂, C₂₋₆alkenyleneN(R^{a})₂, C(═O)NR^{a}C₁₋₄alkyleneOR^{a}, C(═O)NR^{a}C₁₋₄alkyleneHet, OC₂₋₄alkyleneN(R^{a})₂, OC₁₋₄alkyleneCH(OR^{a})CH₂N(R^{a})₂, OC₁₋₄alkyleneHet, OC₂₋₄alkyleneOR^{a}, OC₂₋₄alkyleneNR^{a}C(═O)OR^{a}, NR^{a}C₁₋₄alkyleneN(R^{a})₂, NR^{a}C(═O)R^{a}, NR^{a}C(═O)N(R^{a})₂, N(SO₂C₁₋₄alkyl)₂, NR^{a}(SO₂C₁₋₄alkyl), SO₂N(R^{a})₂, OSO₂CF₃, C₁₋₃alkylenearyl, C₁₋₄alkyleneHet, C₁₋₆alkyleneOR^{a}, C₁₋₃alkyleneN(R^{a})₂, C(═O)N(R^{a})₂, NHC(═O)C₁₋₃alkylenearyl, C₃₋₈cycloalkyl, C₃₋₈heterocycloalkyl, arylOC₁₋₃alkyleneN(R^{a})₂, arylOC(═O)R^{a}, NHC(═O)C₁₋₃alkyleneC₃₋₈heterocycloalkyl, NHC(═O)C₁₋₃alkyleneHet, OC₁₋₄alkyleneOC₁₋₄alkyleneC(═O)OR^{a}, C(═O)C₁₋₄alkyleneHet, and NHC(═O)haloC₁₋₆alkyl, each of which is optionally substituted.

In specific embodiments of Formula III, each R⁶ is independently selected from the group consisting of hydrogen, F, Cl, Br, NO₂, CN, CF₃, and OCF₃, or from the group consisting of methyl, ethyl, propyl, butyl, phenyl, heteroaryl, OR^{a}, N(R^{a})₂, OC(═O)R^{a}, C(═O)R^{a}, C(═O)OR^{a}, Het, each of which is optionally substituted. In some embodiments, R⁶ is selected from H, F, Cl, Br, CN, CF₃, and Me.

In such embodiments of Formula III, n is 0-2. In some embodiments n is 0; in other embodiments, n is 1; and in other embodiments, n is 2. Where n is 1 and R⁶ is not H, it is sometimes preferred for R⁶ to be positioned ortho to the point at which the phenyl ring on which R⁶ is located is attached to the N of the quinoxaline ring.

In such embodiments of Formula III R⁷ is often selected from the group consisting of hydrogen, F, Cl, Br, NO₂, CN, CF₃, and OCF₃, or from the group consisting of methyl, ethyl, propyl, butyl, phenyl, heteroaryl, OR^{a}, N(R^{a})₂, OC(═O)R^{a}, C(═O)R^{a}, C(═O)OR^{a}, Het, each of which is optionally substituted. In some of these embodiments, R⁷ is H, F, Me, CF₃, or NH₂, and preferably R⁷ is attached to a carbon of the 6-membered ring.

In specific embodiments, R⁷ is NH₂. In further specific embodiments, R⁷ is NH₂ at position 2 of the purinyl ring. In further specific embodiments, R⁷ is NH₂ at position 6 of the purinyl ring. In further specific embodiments, R⁷ is NH₂ at position 8 of the purinyl ring.

In such embodiments of Formula III, R¹ and R², independently, are sometimes selected from the group consisting of hydrogen, F, Cl, Br, NO₂, CF₃, OCF₃, and CN, or from the group consisting of methyl, ethyl, propyl, butyl, phenyl, heteroaryl, OR^{a}, N(R^{a})₂, OC(═O)R^{a}, C(═O)R^{a}, C(═O)OR^{a}, each of which is optionally substituted. In some embodiments, H, F, Cl, Br, CN, CF₃, and Me are preferred.

In such embodiments of Formula III, R^{b} is sometimes selected from the group consisting of hydrogen, halo, and CN, or from the group consisting of methyl, ethyl, propyl, butyl, C(═O)R^{a}, and C(═O)OR^{a}, each of which may be optionally substituted; H, Me, Et, and propyl are sometimes preferred.

In such embodiments of Formula III, each R⁶ is sometimes selected from the group consisting of hydrogen, F, Cl, Br, NO₂, CN, CF₃, and OCF₃, or from the group consisting of methyl, ethyl, propyl, butyl, phenyl, heteroaryl, OR^{a}, N(R^{a})₂, OC(═O)R^{a}, C(═O)R^{a}, C(═O)OR^{a}, Het, each of which is optionally substituted; particularly H, F, Cl, or Me; n is 0-2; and R⁷ is selected from the group consisting of hydrogen, F, Cl, Br, NH₂, NO₂, CN, CF₃, and OCF₃, or from the group consisting of methyl, ethyl, propyl, butyl, phenyl, heteroaryl, OR^{a}, N(R^{a})₂, OC(═O)R^{a}, C(═O)R^{a}, C(═O)OR^{a}, Het, each of which is optionally substituted; and H or NH₂ is sometimes preferred.

In specific embodiments, the compound of Formula I is selected from the group consisting of

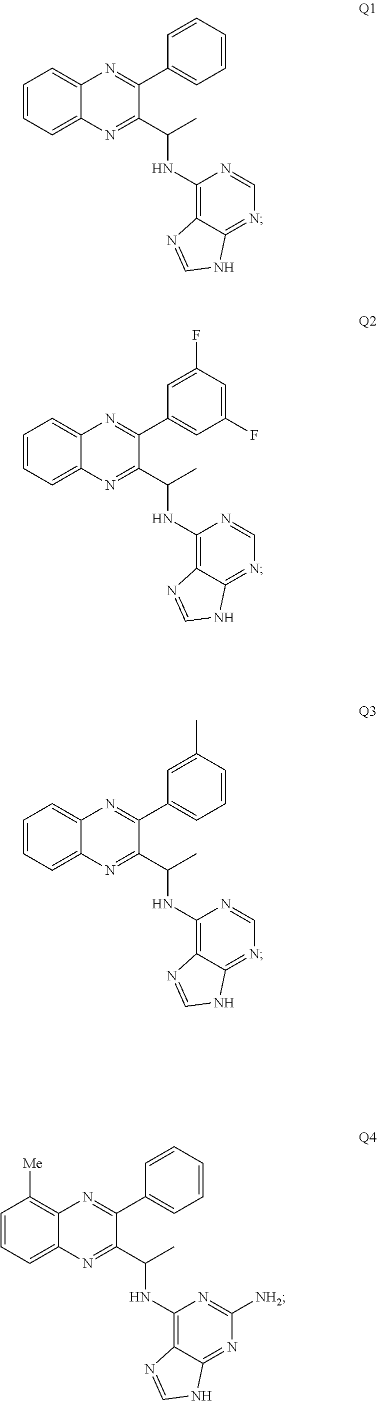 [SMILES: CC(Nc1ncnc2[nH]cnc12)c1nc2ccccc2nc1-c1ccccc1 ; CC(Nc1ncnc2[nH]cnc12)c1nc2ccccc2nc1-c1cc(F)cc(F)c1 ; Cc1cccc(-c2nc3ccccc3nc2C(C)Nc2ncnc3[nH]cnc23)c1 ; Cc1cccc2nc(C(C)Nc3nc(N)nc4[nH]cnc34)c(-c3ccccc3)nc12]

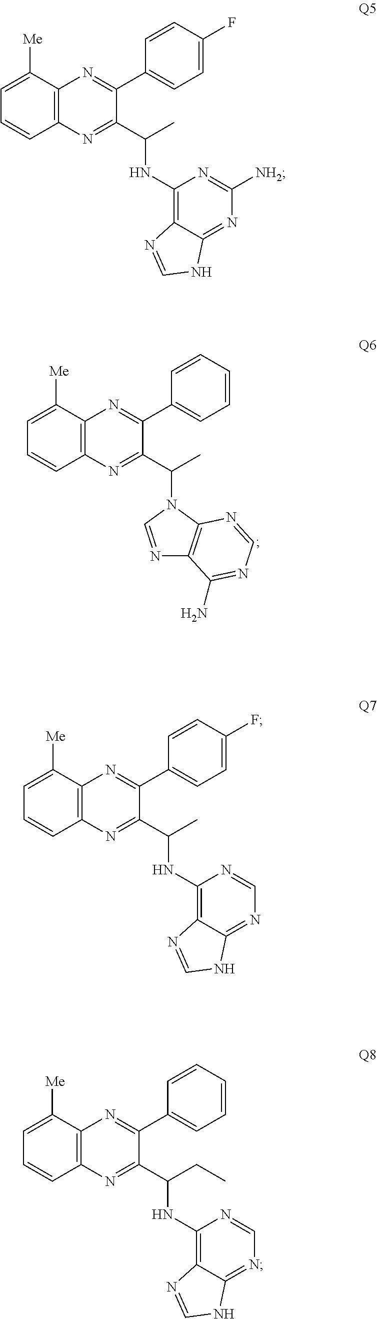 [SMILES: Cc1cccc2nc(C(C)Nc3nc(N)nc4[nH]cnc34)c(-c3ccc(F)cc3)nc12 ; Cc1cccc2nc(C(C)n3cnc4c(N)ncnc43)c(-c3ccccc3)nc12 ; Cc1cccc2nc(C(C)Nc3ncnc4[nH]cnc34)c(-c3ccc(F)cc3)nc12 ; CCC(Nc1ncnc2[nH]cnc12)c1nc2cccc(C)c2nc1-c1ccccc1]

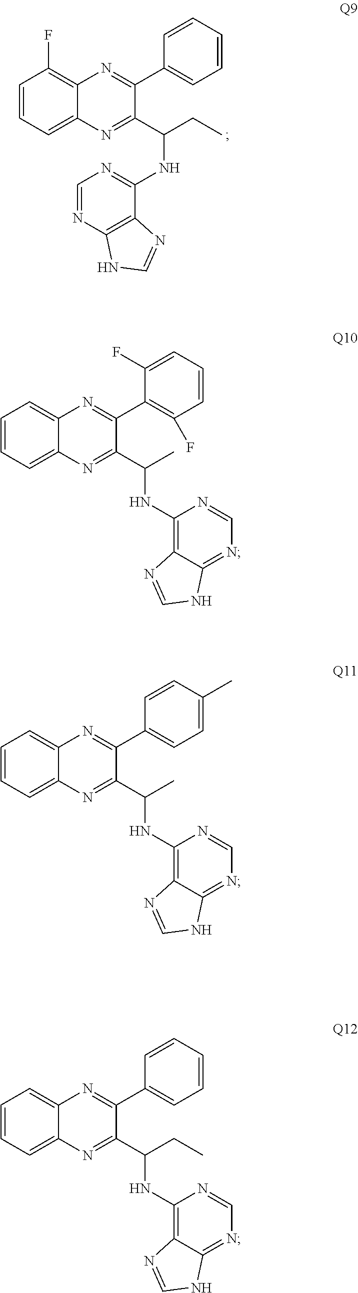 [SMILES: CCC(Nc1ncnc2[nH]cnc12)c1nc2cccc(F)c2nc1-c1ccccc1 ; CC(Nc1ncnc2[nH]cnc12)c1nc2ccccc2nc1-c1c(F)cccc1F ; Cc1ccc(-c2nc3ccccc3nc2C(C)Nc2ncnc3[nH]cnc23)cc1 ; CCC(Nc1ncnc2[nH]cnc12)c1nc2ccccc2nc1-c1ccccc1]

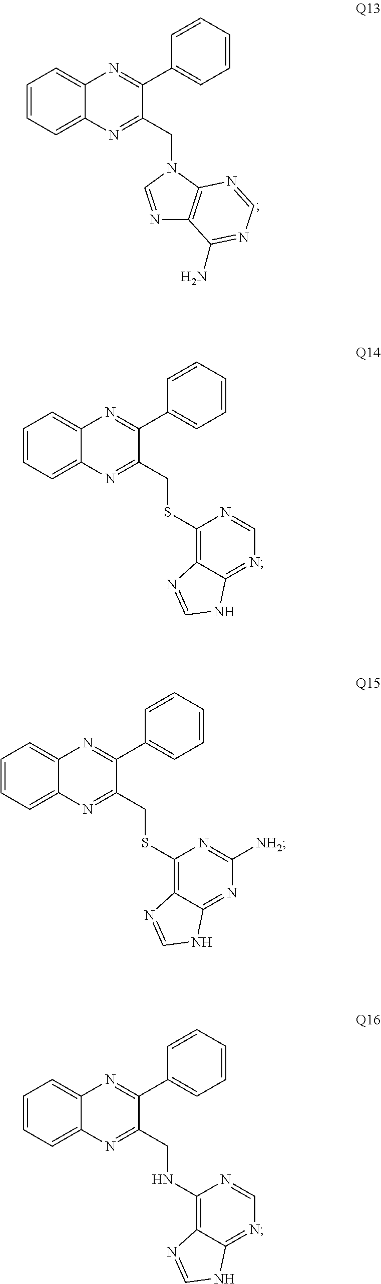 [SMILES: Nc1ncnc2c1ncn2Cc1nc2ccccc2nc1-c1ccccc1 ; c1ccc(-c2nc3ccccc3nc2CSc2ncnc3[nH]cnc23)cc1 ; Nc1nc(SCc2nc3ccccc3nc2-c2ccccc2)c2nc[nH]c2n1 ; c1ccc(-c2nc3ccccc3nc2CNc2ncnc3[nH]cnc23)cc1]

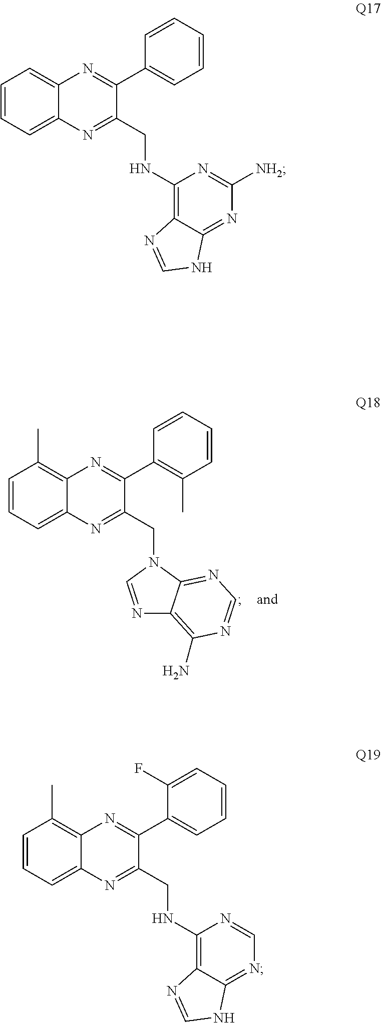 [SMILES: Nc1nc(NCc2nc3ccccc3nc2-c2ccccc2)c2nc[nH]c2n1 ; Cc1ccccc1-c1nc2c(C)cccc2nc1Cn1cnc2c(N)ncnc21 ; Cc1cccc2nc(CNc3ncnc4[nH]cnc34)c(-c3ccccc3F)nc12]

and pharmaceutically acceptable salts thereof.

Compounds Q1-Q12 have a chiral center located in the acyclic linker between the quinoxaline moiety and the purine moiety. In some embodiments, the compound contains a mixture of R and S isomers. In some embodiments the compound is optically active, and in some embodiments it is preferably enriched in the S enantiomer. In some embodiments, such mixture will contain no more than about 10% of the R isomer, meaning the ratio of S to R isomers is at least about 9:1, and preferably less than 5% of the R-isomer, meaning the ratio of S to R enantiomers is at least about 19:1. In some embodiments the compound has less than 2% R enantiomer, meaning it has an enantiomeric excess of at least about 96%. In some embodiments, the compound has an enantiomeric excess of at least 98%. In some embodiments, the compound has an enantiomeric excess of at least 99%.

In some instances, the compounds of the invention can exhibit atropisomerism, where there is restricted rotation between the phenyl ring on the quinoxaline N in Formula II or III, for example, and the quinoxaline ring. This occurs especially when an ortho substituent larger than H, e.g., Cl or Me, is present on that phenyl ring. In such compounds, the invention includes mixtures of atropisomers as well as the individual atropisomers, which can generally be separated by conventional means, such as chiral chromatography.

Some of the compounds of the invention can exist in multiple tautomeric forms, and some of the compounds of the invention include other chiral centers besides the one on the linker between the two ring systems of Formula I. The invention includes each such tautomer and each isomer individually, as well as mixtures thereof.

In many cases, the compounds of the invention conveniently form salts, and the invention includes the neutral compounds as well as their conventional salts. Pharmaceutically acceptable salts, in particular, are included and can be used in methods or compositions where the use of a compound of any of formulas I-III is described herein. Suitable pharmaceutically acceptable salts are further described below.

In another aspect, the invention provides a method to prevent or treat a condition in a subject in need thereof, wherein said condition is an inflammatory condition or cancer, comprising administering to the subject a therapeutically effective amount of a compound described herein.

Examples of inflammatory conditions include arthritic diseases, such as rheumatoid arthritis, psoriatic arthritis, monoarticular arthritis, osteoarthritis, gouty arthritis, spondylitis; Behçet disease; sepsis, septic shock, endotoxic shock, gram negative sepsis, gram positive sepsis, and toxic shock syndrome; multiple organ injury syndrome secondary to septicemia, trauma, or hemorrhage; ophthalmic disorders such as allergic conjunctivitis, vernal conjunctivitis, uveitis, and thyroid-associated ophthalmopathy; eosinophilic granuloma; pulmonary or respiratory disorders such as asthma, chronic bronchitis, allergic rhinitis, acute respiratory distress syndrome (ARDS), chronic pulmonary inflammatory disease (e.g., chronic obstructive pulmonary disease), silicosis, pulmonary sarcoidosis, pleurisy, alveolitis, vasculitis, emphysema, pneumonia, bronchiectasis, and pulmonary oxygen toxicity; reperfusion injury of the myocardium, brain, or extremities; fibrosis such as cystic fibrosis; keloid formation or scar tissue formation; atherosclerosis; autoimmune diseases, such as systemic lupus erythematosus (SLE), autoimmune thyroiditis, multiple sclerosis, some forms of diabetes, and Reynaud's syndrome; and transplant rejection disorders such as GVHD and allograft rejection; chronic glomerulonephritis; inflammatory bowel diseases such as chronic inflammatory bowel disease (CIBD), Crohn's disease, ulcerative colitis, and necrotizing enterocolitis; inflammatory dermatoses such as contact dermatitis, atopic dermatitis, psoriasis, or urticaria; fever and myalgias due to infection; central or peripheral nervous system inflammatory disorders such as meningitis, encephalitis, and brain or spinal cord injury due to minor trauma; Sjogren's syndrome; diseases involving leukocyte diapedesis; alcoholic hepatitis; bacterial pneumonia; antigen-antibody complex mediated diseases; hypovolemic shock; Type I diabetes mellitus; acute and delayed hypersensitivity; disease states due to leukocyte dyscrasia and metastasis; thermal injury; granulocyte transfusion-associated syndromes; and cytokine-induced toxicity.

In some embodiments, the condition is an inflammatory condition, wherein the inflammatory condition is selected from the group consisting of arthritic diseases, ophthalmic disorders, autoimmune diseases, transplant rejection disorders, and inflammatory bowel diseases.

“Autoimmune disease” as used herein refers to any group of disorders in which tissue injury is associated with humoral or cell-mediated responses to the body's own constituents.

In some embodiments, the condition is an inflammatory condition, wherein the inflammatory condition is selected from the group consisting of rheumatoid arthritis, psoriatic arthritis, monoarticular arthritis, osteoarthritis, gouty arthritis, spondylitis, Behçet disease, sepsis, septic shock, endotoxic shock, gram negative sepsis, gram positive sepsis, and toxic shock syndrome, multiple organ injury syndrome secondary to septicemia, trauma, or hemorrhage, allergic conjunctivitis, vernal conjunctivitis, uveitis, thyroid-associated ophthalmopathy, eosinophilic granuloma, asthma, chronic bronchitis, allergic rhinitis, acute respiratory distress syndrome (ARDS), chronic obstructive pulmonary disease (COPD), silicosis, pulmonary sarcoidosis, pleurisy, alveolitis, vasculitis, emphysema, pneumonia, bronchiectasis, pulmonary oxygen toxicity, reperfusion injury of the myocardium, brain, or extremities, cystic fibrosis, keloid formation, scar tissue formation, atherosclerosis, systemic lupus erythematosus (SLE), autoimmune thyroiditis, multiple sclerosis, diabetes, Reynaud's syndrome, graft-versus-host-disease (GVHD), allograft rejection, chronic glomerulonephritis, chronic inflammatory bowel disease (CIBD), Crohn's disease, ulcerative colitis, necrotizing enterocolitis, contact dermatitis, atopic dermatitis, psoriasis, or urticaria, fever, myalgias due to infection, meningitis, encephalitis, brain or spinal cord injury due to minor trauma, Sjogren's syndrome, diseases involving leukocyte diapedesis, alcoholic hepatitis, bacterial pneumonia, antigen-antibody complex mediated diseases, hypovolemic shock, Type I diabetes mellitus, acute and delayed hypersensitivity, disease states due to leukocyte dyscrasia and metastasis, thermal injury, granulocyte transfusion-associated syndromes, and cytokine-induced toxicity.

The method can have utility in treating subjects who are or can be subject to reperfusion injury, i.e., injury resulting from situations in which a tissue or organ experiences a period of ischemia followed by reperfusion. The term “ischemia” refers to localized tissue anemia due to obstruction of the inflow of arterial blood. Transient ischemia followed by reperfusion characteristically results in neutrophil activation and transmigration through the endothelium of the blood vessels in the affected area. Accumulation of activated neutrophils in turn results in generation of reactive oxygen metabolites, which damage components of the involved tissue or organ. This phenomenon of “reperfusion injury” is commonly associated with conditions such as vascular stroke (including global and focal ischemia), hemorrhagic shock, myocardial ischemia or infarction, organ transplantation, and cerebral vasospasm. To illustrate, reperfusion injury occurs at the termination of cardiac bypass procedures or during cardiac arrest when the heart, once prevented from receiving blood, begins to reperfuse.

In some embodiments, the condition is cancer. In a particular embodiment, the cancer is a hematological malignancy and/or solid tumor. In another particular embodiment, the hematological malignancy is leukemia or lymphoma. In some embodiments, lymphoma is a mature (peripheral) B-cell neoplasm. In specific embodiments, the mature B-cell neoplasm is selected from the group consisting of B-cell chronic lymphocytic leukemia/small lymphocytic lymphoma; B-cell prolymphocytic leukemia; Lymphoplasmacytic lymphoma; Splenic marginal zone B-cell lymphoma (+/−villous lymphocytes); Nodal marginal zone lymphoma (+/−monocytoid B-cells); Extranodal marginal zone B-cell lymphoma of mucosa-associated lymphoid tissue (MALT) type; Hairy cell leuekmia; Plasma cell myeloma/plasmacytoma; Follicular lymphoma, follicle center; Mantle cell lymphoma; Diffuse large cell B-cell lymphoma (including Mediastinal large B-cell lymphoma, Intravascular large B-cell lymphoma, and Primary effusion lymphoma); and Burkitt's lymphoma/Burkitt's cell leukemia. In some embodiments, lymphoma is selected from the group consisting of multiple myeloma (MM) and non-Hodgkin's lymphoma (NHL), mantle cell lymphoma (MCL), follicular lymphoma, Waldenstrom's macroglobulinemia (WM) or B-cell lymphoma and diffuse large B-cell lymphoma (DLBCL).

In a further particular embodiment, leukemia is selected from the group consisting of acute lymphocytic leukemia (ALL), acute myeloid leukemia (AML), chronic lymphocytic leukemia (CLL), and small lymphocytic lymphoma (SLL). Acute lymphocytic leukemia is also known as acute lymphoblastic leukemia and may be used interchangeably herein. Both terms describe a type of cancer that starts from the white blood cells, lymphocytes, in the bone marrow.

In some embodiments, Non-Hodgkin's Lymphoma (NHL) falls into one of two categories, aggressive NHL or indolent NHL. Aggressive NHL is fast growing and may lead to a patient's death relatively quickly. Untreated survival may be measured in months or even weeks. Examples of aggressive NHL includes B-cell neoplasms, diffuse large B-cell lymphoma, T/NK cell neoplasms, anaplastic large cell lymphoma, peripheral T-cell lymphomas, precursor B-lymphoblastic leukemia/lymphoma, precursor T-lymphoblastic leukemia/lymphoma, Burkitt's lymphoma, Adult T-cell lymphoma/leukemia (HTLV1+), primary CNS lymphoma, mantle cell lymphoma, polymorphic post-transplantation lymphoproliferative disorder (PTLD), AIDS-related lymphoma, true histiocytic lymphoma, and blastic NK-cell lymphoma. The most common type of aggressive NHL is diffuse large cell lymphoma.

Indolent NHL, on the other hand, is slow growing and does not display obvious symptoms for most patients until the disease has progressed to an advanced stage. Untreated survival of patients with indolent NHL may be measured in years. Non-limiting examples include follicular lymphoma, small lymphocytic lymphoma, extranodal marginal zone lymphoma (also called mucosa associated lymphoid tissue—MALT lymphoma), nodal marginal zone B-cell lymphoma (monocytoid B-cell lymphoma), splenic marginal zone lymphoma, and lymphoplasmacytic lymphoma (Waldenstrom's macroglobulinemia).

In some cases, histologic transformation may occur, e.g., indolent NHL in patients may convert to aggressive NHL.

In some embodiments, the invention provides methods of treating a patient with aggressive NHL or indolent NHL.

In some embodiments, the invention provides methods of treating a patient with a hematological malignancy selected from the group consisting of acute lymphocytic leukemia (ALL), acute myeloid leukemia (AML), chronic lymphocytic leukemia (CLL), multiple myeloma (MM), and non-Hodgkin lymphoma (NHL). In certain embodiments, the non-Hodgkin lymphoma is selected from the group consisting of large diffuse B-cell lymphoma (LDBCL), mantle cell lymphoma (MCL), Waldenstrom's macroglobulinemia (WM) and lymphoplasmacytic lymphoma.

In other embodiments, the cancer is a solid tumor. Examples of solid tumors include myxoid and round cell carcinomas, human soft tissue sarcomas, cancer metastases, squamous cell carcinomas, esophageal squamous cell carcinomas, oral carcinomas, cancers of the adrenal cortex, ACTH-producing tumors, non-small cell lung cancers, breast cancers, gastrointestinal cancers, pancreatic cancers, liver cancers, urological cancers, malignancies of the female reproductive tract, ovarian cancer, cervical cancer, malignancies of the male reproductive tract, prostate cancer, kidney cancers, brain cancers, such as glioma, anaplastic oligodendroglioma, adult glioblastoma multiforme, and adult anaplastic astrocytoma, bone cancers, skin cancers, melanoma, thyroid cancers, retinoblastomas, neuroblastomas, peritoneal effusions, malignant pleural effusions, mesotheliomas, Wilms tumors, gall bladder cancers, trophoblastic neoplasms, hemangiopericytomas, Kaposi's sarcomas, and neuroendocrine cancer.

The methods of the invention comprise administering any of the compounds described herein, such as a compound of Formula I, II, or III, or any of compounds Q1-Q19. In specific embodiments, the compound is predominantly the S-enantiomer.

In yet another aspect, the invention provides for a pharmaceutical composition comprising any compound described herein; and at least one pharmaceutically acceptable excipient. In specific embodiments, the pharmaceutical composition comprises a chiral center in the noncyclic linking group between the quinoxaline moiety and the purine moiety. In further specific embodiments, the S-enantiomer of the compound predominates over the R enantiomer by a ratio of at least about 9:1.

As used herein, the term “alkyl” is defined as straight chained or branched hydrocarbon groups or cyclic hydrocarbon groups containing the indicated number of carbon atoms. Non-limiting examples include methyl, ethyl, and straight chain and branched propyl and butyl groups. Examples of cyclic hydrocarbon groups include cyclopropyl, cyclopentyl and cyclohexyl groups. Alkyl also includes combinations of straight chain, branched chain and cyclic groups, e.g., cyclopropylmethyl and norbornyl. The hydrocarbon group can contain up to 16 carbon atoms, preferably one to eight carbon atoms. The term “alkyl” includes cyclic, bicyclic, and “bridged alkyl,” i.e., a C6-C16 bicyclic or polycyclic hydrocarbon group, for example, norbornyl, adamantyl, bicyclo[2.2.2]octyl, bicyclo[2.2.1]heptyl, bicyclo[3.2.1]octyl, or decahydronaphthyl. The term “cycloalkyl” is defined as a cyclic C3-C8 hydrocarbon group, e.g., cyclopropyl, cyclobutyl, cyclohexyl, and cyclopentyl.

The term “alkenyl” is defined identically as “alkyl,” except the hydrocarbon groups contain at least two carbons and at least one carbon-carbon double bond. The term “alkynyl” defined identically as “alkyl,” except the hydrocarbon groups contain at least two carbons and at least one carbon-carbon triple bond. “Cycloalkenyl” is defined similarly to cycloalkyl, except at least one carbon-carbon double bond is present in the ring.

The term “perfluoroalkyl” is defined as an alkyl group wherein each hydrogen atom is replaced by fluorine.

The term “alkylene” is defined as an alkyl group having a substituent, for example, the term “C1-3alkylenearyl” refers to an alkyl group containing one to three carbon atoms, and substituted with an aryl group. Similarly, “alkylene” when used without description of another group can refer to a divalent alkyl group, which can link two other structural features together, for example, CH₂ and (CH₂)₃ are 1-carbon and 3-carbon alkylene groups.

The term “halo” or “halogen” is defined herein to include fluorine, bromine, chlorine, and iodine. Often, fluoro or chloro is preferred.

The term “haloalkyl” is defined herein as an alkyl group substituted with one or more halo substituents, either fluoro, chloro, bromo, iodo, or combinations thereof. Similarly, “halocycloalkyl” is defined as a cycloalkyl group having one or more halo substituents.

The term “aryl,” alone or in combination, is defined herein as a monocyclic or polycyclic aromatic group, preferably a monocyclic or bicyclic aromatic group, e.g., phenyl or naphthyl. Unless otherwise indicated, an “aryl” group can be unsubstituted or substituted, for example, with one or more, and in particular one to three, halo, alkyl, phenyl, hydroxyalkyl, alkoxy, alkoxyalkyl, haloalkyl, nitro, amino, alkylamino, acylamino, alkylthio, alkylsulfinyl, and alkylsulfonyl. In some embodiments, specific substituents include F, Br, Cl, methyl, ethyl, propyl, isopropyl, and NH₂. Exemplary aryl groups include phenyl, naphthyl, biphenyl, tetrahydronaphthyl, chlorophenyl, fluorophenyl, aminophenyl, methylphenyl, methoxyphenyl, trifluoromethylphenyl, nitrophenyl, carboxyphenyl, and the like.

The term “heteroaryl” is defined herein as a monocyclic or bicyclic ring system containing one or two aromatic rings and containing at least one nitrogen, oxygen, or sulfur atom in an aromatic ring and up to three such heteroatoms per ring, and which can be unsubstituted or substituted, for example, with one or more, and in particular one to three, substituents, like halo, alkyl, hydroxy, hydroxyalkyl, alkoxy, alkoxyalkyl, haloalkyl, nitro, amino, alkylamino, acylamino, alkylthio, alkylsulfinyl, and alkylsulfonyl. Examples of heteroaryl groups include purinyl, thienyl, furyl, pyridyl, oxazolyl, quinolyl, isoquinolyl, indolyl, triazolyl, isothiazolyl, isoxazolyl, imidazolyl, benzothiazolyl, pyrazinyl, pyrimidinyl, thiazolyl, and thiadiazolyl.

The term “C3-8heterocycloalkyl” is defined as monocyclic ring system containing one or more heteroatoms selected from the group consisting of oxygen, nitrogen, and sulfur. A “C3-8heterocycloalkyl” group also can contain an oxo group (═O) attached to the ring. Nonlimiting examples of “C3-8heterocycloalkyl” groups include 1,3-dioxolane, 2-pyrazoline, pyrazolidine, pyrrolidine, piperazine, a pyrroline, 2H-pyran, 4H-pyran, morpholine, thiopholine, piperidine, 1,4-dithiane, and 1,4-dioxane.

The term “hydroxy” is defined as —OH.

The term “alkoxy” is defined as —OR, wherein R is C1-C8 alkyl, C2-C8 alkenyl or C2-C8 alkynyl; each alkyl, alkenyl and alkynyl group is optionally substituted.

The term “alkoxyalkyl” is defined as an alkyl group wherein a hydrogen has been replaced by an alkoxy group. The term “(alkylthio)alkyl” is defined similarly as alkoxyalkyl, except a sulfur atom, rather than an oxygen atom, is present.

The term “hydroxyalkyl” is defined as a hydroxy group appended to an alkyl group.

The term “alkylthio” is defined as —SR, wherein R is alkyl.

The term “alkylsulfinyl” is defined as R—SO, wherein R is alkyl.

The term “alkylsulfonyl” is defined as R—SO₂, wherein R is alkyl.

The term “amino” is defined as —NH₂, and the term “alkylamino” is defined as —NR₂, wherein at least one R is alkyl, alkenyl or alkynyl, and the second R is alkyl, alkenyl, alkynyl or hydrogen.

The term “acylamino” is defined as RC(═O)N, wherein R is alkyl, alkenyl, alkynyl or aryl, heteroaryl, or heterocyclyl.

The term “nitro” is defined as —NO₂.

The term “trifluoromethyl” is defined as —CF₃.

The term “trifluoromethoxy” is defined as —OCF₃.

The term “cyano” is defined as —CN.

Alkyl, alkenyl and alkynyl groups are often substituted to the extent that such substitution makes sense chemically. Typical substituents include, but are not limited to, halo, ═O, ═N—CN, ═N—OR, ═NR, OR, NR₂, SR, SO₂R, SO₂NR₂, NRSO₂R, NRCONR₂, NRCOOR, NRCOR, CN, COOR, CONR₂, OOCR, COR, and NO₂, wherein each R is independently H, C1-C8 alkyl, C2-C8 heteroalkyl, C1-C8 acyl, C2-C8 heteroacyl, C2-C8 alkenyl, C2-C8 heteroalkenyl, C2-C8 alkynyl, C2-C8 heteroalkynyl, C6-C10 aryl, or C5-C10 heteroaryl, and each R is optionally substituted with halo, ═O, ═N—CN, ═N—OR′, ═NR′, OR′, NR′₂, SR′, SO₂R′, SO₂NR′₂, NR′SO₂R′, NR′CONR′₂, NR′COOR′, NR′COR′, CN, COOR′, CONR′₂, OOCR′, COR′, and NO₂, wherein each R′ is independently H, C1-C8 alkyl, C2-C8 heteroalkyl, C1-C8 acyl, C2-C8 heteroacyl, C6-C10 aryl or C5-C10 heteroaryl. Alkyl, alkenyl and alkynyl groups can also be substituted by C1-C8 acyl, C2-C8 heteroacyl, C6-C10 aryl or C5-C10 heteroaryl, each of which can be substituted by the substituents that are appropriate for the particular group.

Aryl and heteroaryl moieties may be substituted with a variety of substituents including C1-C8 alkyl, C2-C8 alkenyl, C2-C8 alkynyl, C5-C12 aryl, C1-C8 acyl, and heteroforms of these, each of which can itself be further substituted; other substituents for aryl and heteroaryl moieties include halo, OR, NR₂, SR, SO₂R, SO₂NR₂, NRSO₂R, NRCONR₂, NRCOOR, NRCOR, CN, COOR, CONR₂, OOCR, COR, and NO₂, wherein each R is independently H, C1-C8 alkyl, C2-C8 heteroalkyl, C2-C8 alkenyl, C2-C8 heteroalkenyl, C2-C8 alkynyl, C2-C8 heteroalkynyl, C6-C10 aryl, C5-C10 heteroaryl, C7-C12 arylalkyl, or C6-C12 heteroarylalkyl, and each R is optionally substituted as described above for alkyl groups. The substituent groups on an aryl or heteroaryl group may of course be further substituted with the groups described herein as suitable for each type of such substituents or for each component of the substituent. Thus, for example, an arylalkyl substituent may be substituted on the aryl portion with substituents described herein as typical for aryl groups, and it may be further substituted on the alkyl portion with substituents described herein as typical or suitable for alkyl groups.

“Heteroforms” as used herein refers to a modified alkyl, alkenyl, aryl, etc., wherein at least one heteroatom selected from N, O and S replaces at least one carbon atom in the hydrocarbon group being described. Typically a heteroform has only one such heteroatom replacing one carbon atom.

In a particular embodiment, the subject is a human subject. In a particular embodiment, the subject is refractory to chemotherapy treatment, or in relapse after treatment with chemotherapy. In an alternative embodiment, the subject is a de novo patient.

The compounds of the invention may be formulated for administration to animal subject using commonly understood formulation techniques well known in the art. Formulations which are suitable for particular modes of administration and for the compounds of Formula I may be found in Remington's Pharmaceutical Sciences, latest edition, Mack Publishing Company, Easton, Pa.

A compound of the present invention can be administered as the neat chemical, but it is typically preferable to administer the compound in the form of a pharmaceutical composition or formulation. Accordingly, the present invention also provides pharmaceutical compositions that comprise a compound of Formula I and a biocompatible pharmaceutical carrier, adjuvant, or vehicle. The composition can include the agent as the only active moiety or in combination with other agents, such as oligo- or polynucleotides, oligo- or polypeptides, drugs, or hormones mixed with excipient(s) or other pharmaceutically acceptable carriers. Carriers and other ingredients can be deemed pharmaceutically acceptable insofar as they are compatible with other ingredients of the formulation and not deleterious to the recipient thereof.

The pharmaceutical compositions are formulated to contain suitable pharmaceutically acceptable carriers, and can optionally comprise excipients and auxiliaries that facilitate processing of the active compounds into preparations that can be used pharmaceutically. The administration modality will generally determine the nature of the carrier. For example, formulations for parenteral administration can comprise aqueous solutions of the active compounds in water-soluble form. Carriers suitable for parenteral administration can be selected from among saline, buffered saline, dextrose, water, and other physiologically compatible solutions. Preferred carriers for parenteral administration are physiologically compatible buffers such as Hank's solution, Ringer's solution, or physiologically buffered saline. For tissue or cellular administration, penetrants appropriate to the particular barrier to be permeated are used in the formulation. Such penetrants are generally known in the art. For preparations comprising proteins, the formulation can include stabilizing materials, such as polyols (e.g., sucrose) and/or surfactants (e.g., nonionic surfactants), and the like.

Alternatively, formulations for parenteral use can comprise dispersions or suspensions of the active compounds prepared as appropriate oily injection suspensions. Suitable lipophilic solvents or vehicles include fatty oils, such as sesame oil, and synthetic fatty acid esters, such as ethyl oleate or triglycerides, or liposomes. Aqueous injection suspensions can contain substances that increase the viscosity of the suspension, such as sodium carboxy-methylcellulose, sorbitol, or dextran. Optionally, the suspension also can contain suitable stabilizers or agents that increase the solubility of the compounds to allow for the preparation of highly concentrated solutions. Aqueous polymers that provide pH-sensitive solubilization and/or sustained release of the active agent also can be used as coatings or matrix structures, e.g., methacrylic polymers, such as the Eudragit™ series available from Rohm America Inc. (Piscataway, N.J.). Emulsions, e.g., oil-in-water and water-in-oil dispersions, also can be used, optionally stabilized by an emulsifying agent or dispersant (surface active materials; surfactants). Suspensions can contain suspending agents such as ethoxylated isostearyl alcohols, polyoxyethlyene sorbitol and sorbitan esters, microcrystalline cellulose, aluminum metahydroxide, bentonite, agar-agar, gum tragacanth, and mixtures thereof.

Liposomes containing the active agent also can be employed for parenteral administration. Liposomes generally are derived from phospholipids or other lipid substances. The compositions in liposome form also can contain other ingredients, such as stabilizers, preservatives, excipients, and the like. Preferred lipids include phospholipids and phosphatidyl cholines (lecithins), both natural and synthetic. Methods of forming liposomes are known in the art. See, e.g., Prescott (Ed.), Methods in Cell Biology, Vol. XIV, p. 33, Academic Press, New York (1976).

The pharmaceutical compositions comprising the agent in dosages suitable for oral administration can be formulated using pharmaceutically acceptable carriers well known in the art. The preparations formulated for oral administration can be in the form of tablets, pills, capsules, cachets, dragees, lozenges, liquids, gels, syrups, slurries, elixirs, suspensions, or powders. To illustrate, pharmaceutical preparations for oral use can be obtained by combining the active compounds with a solid excipient, optionally grinding the resulting mixture, and processing the mixture of granules, after adding suitable auxiliaries if desired, to obtain tablets or dragée cores. Oral formulations can employ liquid carriers similar in type to those described for parenteral use, e.g., buffered aqueous solutions, suspensions, and the like.

Preferred oral formulations include tablets, dragees, and gelatin capsules. These preparations can contain one or excipients, which include, without limitation:

a) diluents, such as sugars, including lactose, dextrose, sucrose, mannitol, or sorbitol;

b) binders, such as magnesium aluminum silicate, starch from corn, wheat, rice, potato, etc.;

c) cellulose materials, such as methylcellulose, hydroxypropylmethyl cellulose, and sodium carboxymethylcellulose, polyvinylpyrrolidone, gums, such as gum arabic and gum tragacanth, and proteins, such as gelatin and collagen;

d) disintegrating or solubilizing agents such as cross-linked polyvinyl pyrrolidone, starches, agar, alginic acid or a salt thereof, such as sodium alginate, or effervescent compositions;

e) lubricants, such as silica, talc, stearic acid or its magnesium or calcium salt, and polyethylene glycol;

f) flavorants and sweeteners;

g) colorants or pigments, e.g., to identify the product or to characterize the quantity (dosage) of active compound; and

h) other ingredients, such as preservatives, stabilizers, swelling agents, emulsifying agents, solution promoters, salts for regulating osmotic pressure, and buffers.

In some preferred oral formulations, the pharmaceutical composition comprises at least one of the materials from group (a) above, or at least one material from group (b) above, or at least one material from group (c) above, or at least one material from group (d) above, or at least one material from group (e) above. Preferably, the composition comprises at least one material from each of two groups selected from groups (a)-(e) above.

Gelatin capsules include push-fit capsules made of gelatin, as well as soft, sealed capsules made of gelatin and a coating such as glycerol or sorbitol. Push-fit capsules can contain the active ingredient(s) mixed with fillers, binders, lubricants, and/or stabilizers, etc. In soft capsules, the active compounds can be dissolved or suspended in suitable fluids, such as fatty oils, liquid paraffin, or liquid polyethylene glycol with or without stabilizers.

Dragée cores can be provided with suitable coatings such as concentrated sugar solutions, which also can contain gum arabic, talc, polyvinyl pyrrolidone, carbopol gel, polyethylene glycol, and/or titanium dioxide, lacquer solutions, and suitable organic solvents or solvent mixtures.

The pharmaceutical composition can be provided as a salt of the active agent. Salts tend to be more soluble in aqueous or other protonic solvents than the corresponding free acid or base forms. Pharmaceutically acceptable salts are well known in the art. Compounds that contain acidic moieties can form pharmaceutically acceptable salts with suitable cations. Suitable pharmaceutically acceptable cations include, for example, alkali metal (e.g., sodium or potassium) and alkaline earth (e.g., calcium or magnesium) cations.

Compounds of structural formula (I) that contain basic moieties can form pharmaceutically acceptable acid addition salts with suitable acids. For example, Berge, et al., describe pharmaceutically acceptable salts in detail in J. Pharm. Sci. (1977) 66:1. The salts can be prepared in situ during the final isolation and purification of the compounds of the invention or separately by reacting a free base function with a suitable acid.

Representative acid addition salts include, but are not limited to, acetate, adipate, alginate, citrate, aspartate, benzoate, benzenesulfonate, bisulfate, butyrate, camphorate, camphorolsulfonate, digluconate, glycerophosphate, hemisulfate, heptanoate, hexanoate, fumarate, hydrochloride, hydrobromide, hydroiodide, 2-hydroxyethanesulfonate (isothionate), lactate, maleate, methanesulfonate or sulfate, nicotinate, 2-naphthalenesulfonate, oxalate, pamoate, pectinate, persulfate, 3-phenylpropionate, picrate, pivalate, propionate, succinate, tartrate, thiocyanate, phosphate or hydrogen phosphate, glutamate, bicarbonate, p-toluenesulfonate, and undecanoate. Examples of acids that can be employed to form pharmaceutically acceptable acid addition salts include, without limitation, such inorganic acids as hydrochloric acid, hydrobromic acid, sulfuric acid, and phosphoric acid, and such organic acids as oxalic acid, maleic acid, succinic acid, and citric acid.

Basic nitrogen-containing groups can be quaternized with such agents as lower alkyl halides such as methyl, ethyl, propyl, and butyl chlorides, bromides and iodides; dialkyl sulfates like dimethyl, diethyl, dibutyl, and diamyl sulfates; long chain alkyl halides such as decyl, lauryl, myristyl, and stearyl chlorides, bromides, and iodides; arylalkyl halides such as benzyl and phenethyl bromides; and others. Products having modified solubility or dispersibility are thereby obtained.

Compositions comprising a compound of the invention formulated in a pharmaceutical acceptable carrier can be prepared, placed in an appropriate container, and labeled for treatment of an indicated condition. Accordingly, there also is contemplated an article of manufacture, such as a container comprising a dosage form of a compound of the invention and a label containing instructions for use of the compound. Kits are also contemplated under the invention. For example, the kit can comprise a dosage form of a pharmaceutical composition and a package insert containing instructions for use of the composition in treatment of a medical condition. In either case, conditions indicated on the label can include treatment of inflammatory disorders, cancer, etc.

Methods of Administration

Pharmaceutical compositions comprising a compound of Formula I can be administered to the subject by any conventional method, including parenteral and enteral techniques. Parenteral administration modalities include those in which the composition is administered by a route other than through the gastrointestinal tract, for example, intravenous, intraarterial, intraperitoneal, intramedullarly, intramuscular, intraarticular, intrathecal, and intraventricular injections. Enteral administration modalities include, for example, oral (including buccal and sublingual) and rectal administration. Transepithelial administration modalities include, for example, transmucosal administration and transdermal administration. Transmucosal administration includes, for example, enteral administration as well as nasal, inhalation, and deep lung administration; vaginal administration; and rectal administration. Transdermal administration includes passive or active transdermal or transcutaneous modalities, including, for example, patches and iontophoresis devices, as well as topical application of pastes, salves, or ointments. Parenteral administration also can be accomplished using a high-pressure technique, e.g., Powderject™.

Surgical techniques include implantation of depot (reservoir) compositions, osmotic pumps, and the like. A preferred route of administration for treatment of inflammation can be local or topical delivery for localized disorders such as arthritis, or systemic delivery for distributed disorders, e.g., intravenous delivery for reperfusion injury or for systemic conditions such as septicemia. For other diseases, including those involving the respiratory tract, e.g., chronic obstructive pulmonary disease, asthma, and emphysema, administration can be accomplished by inhalation or deep lung administration of sprays, aerosols, powders, and the like.

The compound of Formula I can be administered before, during, or after administration of chemotherapy, radiotherapy, and/or surgery. The formulation and route of administration chosen will be tailored to the individual subject, the nature of the condition to be treated in the subject, and generally, the judgment of the attending practitioner.

The therapeutic index of the compound of Formula I can be enhanced by modifying or derivatizing the compounds for targeted delivery to cancer cells expressing a marker that identifies the cells as such. For example, the compounds can be linked to an antibody that recognizes a marker that is selective or specific for cancer cells, so that the compounds are brought into the vicinity of the cells to exert their effects locally, as previously described (see for example, Pietersz, et al., Immunol. Rev. (1992) 129:57; Trail, et al., Science (1993) 261:212; and Rowlinson-Busza, et al., Curr. Opin. Oncol. (1992) 4:1142). Tumor-directed delivery of these compounds enhances the therapeutic benefit by, inter alia, minimizing potential nonspecific toxicities that can result from radiation treatment or chemotherapy. In another aspect, the compound of Formula I and radioisotopes or chemotherapeutic agents can be conjugated to the same anti-tumor antibody.

The characteristics of the agent itself and the formulation of the agent can influence the physical state, stability, rate of in vivo release, and rate of in vivo clearance of the administered agent. Such pharmacokinetic and pharmacodynamic information can be collected through preclinical in vitro and in vivo studies, later confirmed in humans during the course of clinical trials. Thus, for any compound used in the method of the invention, a therapeutically effective dose can be estimated initially from biochemical and/or cell-based assays.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the “therapeutic index,” which typically is expressed as the ratio LD50/ED50. Compounds that exhibit large therapeutic indices, i.e., the toxic dose is substantially higher than the effective dose, are preferred. The data obtained from such cell culture assays and additional animal studies can be used in formulating a range of dosage for human use. The dosage of such compounds lies preferably within a range of circulating concentrations that include the ED50 with little or no toxicity.

For the methods of the invention, any effective administration regimen regulating the timing and sequence of doses can be used. Doses of the agent preferably include pharmaceutical dosage units comprising an effective amount of the agent. As used herein, “effective amount” refers to an amount sufficient to modulate the expression of a particular PI3-kinase, such as PI3Kdelta, or activity and/or derive a measurable change in a physiological parameter of the subject through administration of one or more of the pharmaceutical dosage units. “Effective amount” can also refer to the amount required to ameliorate a disease or disorder in a subject.

Suitable dosage ranges for the compounds of Formula I vary according to these considerations, but in general, the compounds are administered in the range of 10.0 μg/kg-15 mg/kg of body weight; 1.0 μg/kg-10 mg/kg of body weight, or 0.5 mg/kg-5 mg/kg of body weight. For a typical 70-kg human subject, thus, the dosage range is from 700 μg-1050 mg; 70 μg-700 mg; or 35 mg-350 mg per dose, and two or more doses may be administered per day. Dosages may be higher when the compounds are administered orally or transdermally as compared to, for example, i.v. administration. In certain embodiments, the treatment of cancers comprises oral administration of up to 750 mg/day of Compound I. The reduced toxicity of this compound permits the therapeutic administration of relatively high doses. For treatment of many solid tumors, a dosage of about 50-100 mg per dose, administered orally once or preferably at least twice per day, is often suitable. In some embodiments, compound I is administered orally, in three to five doses per day, using 20-150 mg per dose for a total daily dose between about 60 and 750 mg. In some embodiments, the total daily dose is between 100 and 500 mg, and in some embodiments the normalized daily dosage (adjusted for subject's body weight) is up to about 60 mg per kg of the treated subject's body weight.

The compounds may be administered as a single bolus dose, a dose over time, as in i.v. or transdermal administration, or in multiple dosages. For i.v. or transdermal delivery, a dosage may be delivered over a prolonged period of time, and may be selected or adjusted to produce a desired plasma level of the active compound. In some embodiments, the desired level will be at least about 1 microM, or at least about 10 microM.

When the compound is administered orally, it is preferably administered in two or more doses per day. In some embodiments, three doses per day are administered. In some embodiments four doses per day are administered.

Dosing may be continued for one day or for multiple days, such as about 7 days. In some embodiments, daily dosing is continued for about 14 days or about 28 days. In some embodiments, dosing is continued for about 28 days and is then discontinued for about 7 days; the efficacy of the treatment can be assessed during the break, when treatment with compound I has been stopped, and if the assessment shows that the treatment is achieving a desired effect, another 7-28 day cycle of treatment with Compound I can be initiated.

Depending on the route of administration, a suitable dose can be calculated according to body weight, body surface area, or organ size. The final dosage regimen will be determined by the attending physician in view of good medical practice, considering various factors that modify the action of drugs, e.g., the agent's specific activity, the identity and severity of the disease state, the responsiveness of the patient, the age, condition, body weight, sex, and diet of the patient, and the severity of any infection. Additional factors that can be taken into account include time and frequency of administration, drug combinations, reaction sensitivities, and tolerance/response to therapy. Further refinement of the dosage appropriate for treatment involving any of the formulations mentioned herein is done routinely by the skilled practitioner without undue experimentation, especially in light of the dosage information and assays disclosed, as well as the pharmacokinetic data observed in human clinical trials. Appropriate dosages can be ascertained through use of established assays for determining concentration of the agent in a body fluid or other sample together with dose response data.

The frequency of dosing will depend on the pharmacokinetic parameters of the agent and the route of administration. Dosage and administration are adjusted to provide sufficient levels of the active moiety or to maintain the desired effect. Accordingly, the pharmaceutical compositions can be administered in a single dose, multiple discrete doses, continuous infusion, sustained release depots, or combinations thereof, as required to maintain desired minimum level of the agent. Short-acting pharmaceutical compositions (i.e., short half-life) can be administered once a day or more than once a day (e.g., two, three, or four times a day). Long acting pharmaceutical compositions might be administered every 3 to 4 days, every week, or once every two weeks. Pumps, such as subcutaneous, intraperitoneal, or subdural pumps, can be preferred for continuous infusion.

Subjects that will respond favorably to the method of the invention include medical and veterinary subjects generally, including human patients. Among other subjects for whom the methods of the invention is useful are cats, dogs, large animals, avians such as chickens, and the like. In general, any subject who would benefit from a compound of Formula I is appropriate for administration of the invention method.

Compounds of the invention may be prepared using a number of methods familiar to one of skill in the art. The discussion below is offered to illustrate certain of the diverse methods available for use in assembling the compounds of the invention. However, the discussion is not intended to define the scope of the reactions or reaction sequences that are useful in preparing compounds of the invention. The following are representative examples of synthetic methods that may be use to prepare the compounds of the invention. The present invention will be understood more readily by reference to the following examples, which are provided by way of illustration and are not intended to be limiting of the present invention.

### Example 1

### Preparation of Compound Q2

N-{1-[3-(3,5-difluorophenyl)quinoxalin-2-yl]ethyl}-9H-purin-6-amine

This representative example describes the synthesis of compound Q2, N-{1-[3-(3,5-difluorophenyl)quinoxalin-2-yl]ethyl}-9H-purin-6-amine. The quinoxaline group of this compound is linked to and attached to the 6-position of a purinyl group via a methyl substituted alkylene-NH linker. Shown below is a synthetic scheme followed by a detailed description of each step.

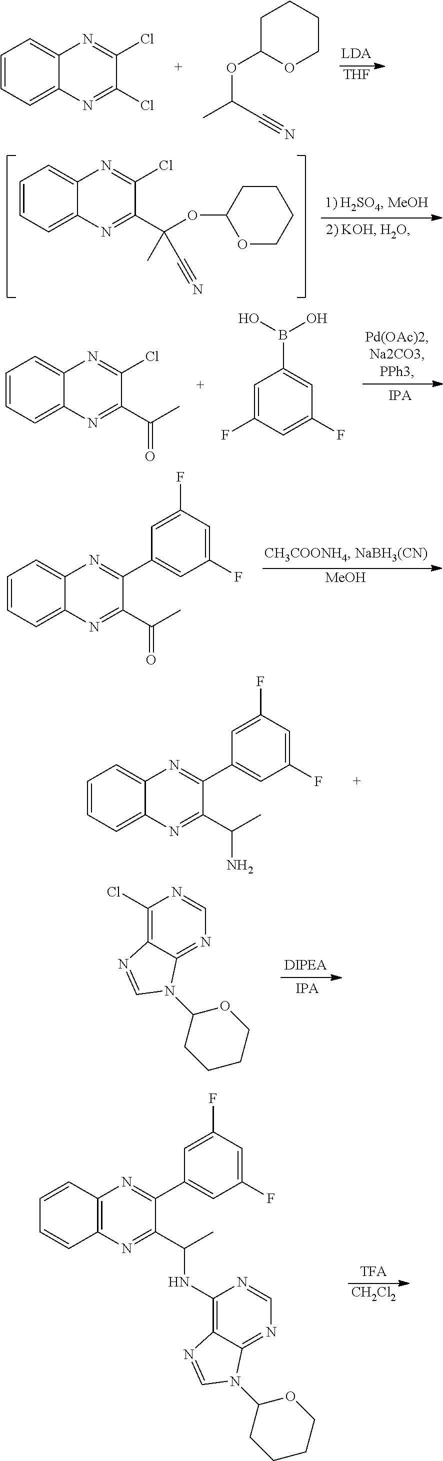

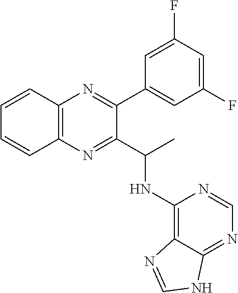 [SMILES: CC(Nc1ncnc2[nH]cnc12)c1nc2ccccc2nc1-c1cc(F)cc(F)c1]

### Step 1: Synthesis of 1-(3-chloroquinoxalin-2-yl)ethanone

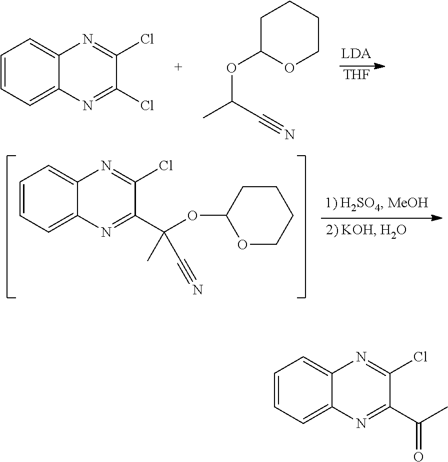

To a flame-dried, 100 mL, round-bottomed flask was added 2-(tetrahydro-2H-pyran-2-yloxy)propanenitrile (3.12 g, 20.1 mmol) (from Org. React., 1984, vol. 31) and 2,3-dichloroquinoxaline (2.0 g, 10.0 mmol) dissolved in dry THF (40 mL). The mixture was stirred and cooled to −78° C. under nitrogen. To the mixture was added a 2.0 M solution of LDA in THF (10 mL) and the reaction was stirred for 1 hour at −78° C., allowed to warm to 0° C. and stirred for an additional 1 hour. Saturated 1N HCl solution (50 mL) was then added and the reaction was allowed to warm to 21° C. then stirred for 18 hours. The solvent was evaporated and the residue was dissolved in methanol (50 mL). 5% Sulfuric acid (5%, 10 mL) was added and the reaction was stirred for 5 hours. The solvent was evaporated and the residue was dissolved in ether (100 mL) and 1N KOH was added until the solution turns basic. The reaction was stirred at 21° C. for 2 hours. The layers were separated and the residue was chromatographed (90 g of silica gel, 1-3% EtOAc in hexanes) to yield 1-(3-chloroquinoxalin-2-yl)ethanone. MS (ESI+) for C₁₀H₇ClN₂O m/z 207.2 (M+H)⁺. ¹H NMR (CDCl₃) δ 8.17 (d, J=8 Hz, 1H), 8.07 (d, J=8 Hz, 1H), 7.917 (m, 2H), 2.846 (s, 3 H).

### Step 2: Synthesis of 1-[3-(3,5-difluorophenyl)quinoxalin-2-yl]ethanone

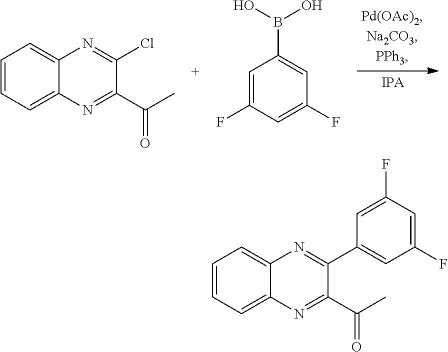 [SMILES: CC(=O)c1nc2ccccc2nc1Cl ; OB(O)c1cc(F)cc(F)c1 ; CC(=O)c1nc2ccccc2nc1-c1cc(F)cc(F)c1]

To 1-(3-chloroquinoxalin-2-yl)ethanone (0.100 g, 0.484 mmol), and 3,5-diphenylboronic acid (0.0841 g, 0.532 mmol) dissolved in 2-propanol (15 mL) was added sodium carbonate (0.06155 g, 0.5808 mmol) and triphenylphosphine (0.0762 g, 0.290 mmol). The reaction mixture was degassed and placed under nitrogen, then palladium acetate (0.0652 g, 0.290 mmol) was added. The reaction was warmed to reflux and stirred for 2 hours. The solvent was evaporated and the residue was dissolved in EtOAc (100 mL), washed with water (1×20 mL) and brine (1×20 mL), dried over sodium sulfate, treated with decolorizing carbon, filtered and concentrated to afford 0.245 g of the crude target as a pale yellow oil which slowly crystallized. This was chromatographed (40 g of silica gel, 10% EtOAc in hexanes) to yield 1-[3-(3,5-difluorophenyl)quinoxalin-2-yl]ethanone as a waxy white solid. MS (ESI+) for C₁₆H₁₀F₂N₂O m/z 285.2 (M+H)⁺. ¹H NMR (CDCl₃) δ 8.21 (m, 2H), 7.92 (m, 2H), 7.16 (m, 2H), 6.95 (m, 1H), 2.87 (s, 3H).

### Step 3: Synthesis of 1-[3-(3,5-difluorophenyl)quinoxalin-2-yl]ethanamine

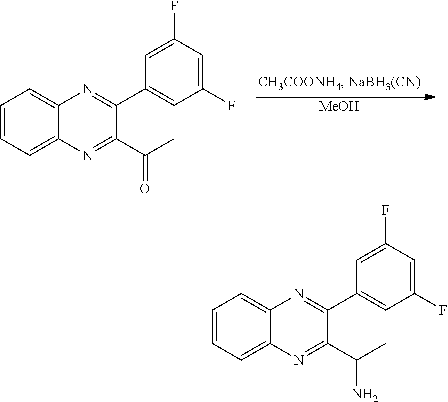 [SMILES: "MeOH" ; CC(=O)c1nc2ccccc2nc1-c1cc(F)cc(F)c1 ; CC(N)c1nc2ccccc2nc1-c1cc(F)cc(F)c1]

To 1-[3-(3,5-difluorophenyl)quinoxalin-2-yl]ethanone (0.20 g, 0.704 mmol) and ammonium acetate (0.542 g, 7.04 mmol) dissolved in MeOH (40 mL) was added sodium cyanoborohydride (0.061 g, 0.985 mmol). The mixture was stirred at 40° C. for 18 hours then stirred at reflux for 9 days. Afterwards, the solvent was evaporated and the residue was dissolved in EtOAc (100 mL), washed with water (1×30 mL) and brine (1×30 mL), dried over sodium sulfate, filtered and concentrated to afford the crude product. The crude was chromatographed (1-5% MeOH containing 10% NH₄OH in chloroform) to afford 1-[3-(3,5-difluorophenyl)quinoxalin-2-yl]ethanamine. MS (ESI+) for C₁₆H₁₃F₂N₃ m/z 286.2 (M+H)⁺. ¹H NMR (CD₃OD) δ8.198 (d, J=2 Hz, 1H), 8.12 (d, J=2 Hz, 1H), 7.88 (m, 2H), 7.365 (m, 2H), 7.21 (m, 1H), 4.92 (s, 2H), 4.48 (q, J=6 Hz, 1H), 1.39 (d, J=6 Hz, 3 H).

### Step 4: Synthesis of N-{1-[3-(3,5-difluorophenyl)quinoxalin-2-yl]ethyl}-9-(tetrahydro-2H-pyran-2-yl)-9H-purin-6-amine

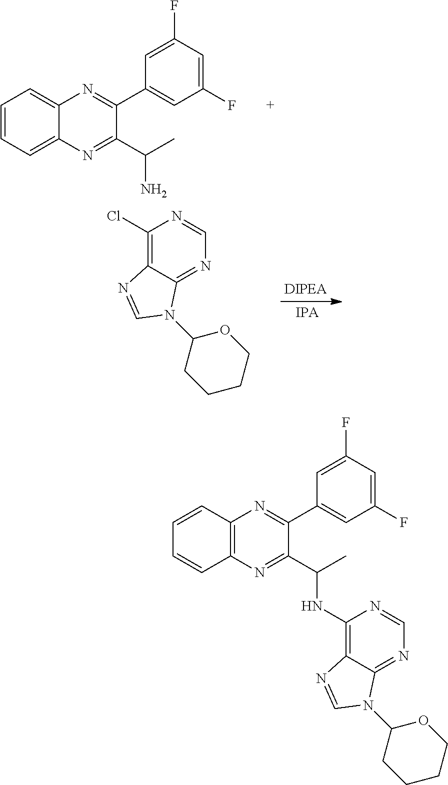

1-[3-(3,5-difluorophenyl)quinoxalin-2-yl]ethanamine (0.078 g, 0.273 mmol), 6-chloro-9-(tetrahydro-2H-pyran-2-yl)-9H-purine (0.0783 g, 0.328 mmol) and DIPEA (57.1 μL, 0.328 mmol) were dissolved in 2-propanol (10 mL) and warmed to 80° C. The reaction was stirred for 48 hours. The solvent was evaporated and the residue was chromatographed (40 g of silica gel, 1-3% MeOH in dichloromethane gradient over 1 L) to yield N-{1-[3-(3,5-difluorophenyl)quinoxalin-2-yl]ethyl}-9-(tetrahydro-2H-pyran-2-yl)-9H-purin-6-amine. MS (ESI+) for C₂₆H₂₃F₂N₇O m/z 488.2 (M+H)⁺. Complex NMR indicated a mixture of diastereomers.

### Step 5: Synthesis of N-{1-[3-(3,5-difluorophenyl)quinoxalin-2-yl]ethyl}-9H-purin-6-amine

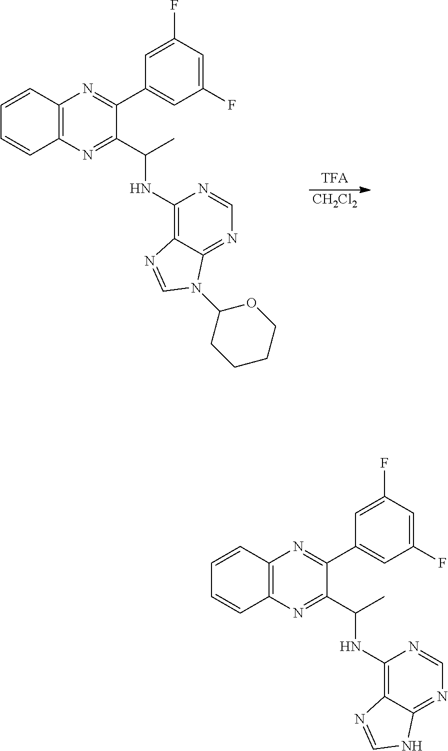 [SMILES: CC(Nc1ncnc2c1ncn2C1CCCCO1)c1nc2ccccc2nc1-c1cc(F)cc(F)c1 ; "CH2Cl2" ; CC(Nc1ncnc2[nH]cnc12)c1nc2ccccc2nc1-c1cc(F)cc(F)c1]

To a solution of N-{1-[3-(3,5-difluorophenyl)quinoxalin-2-yl]ethyl}-9-(tetrahydro-2H-pyran-2-yl)-9H-purin-6-amine (0.233 g, 0.478 mmol) dissolved in dichloromethane (10 mL) was added TFA (200 μL, 2 mmol) and the reaction turns pale yellow. The reaction mixture was stirred at 21° C. for 2 hours. The solvent was evaporated and the residue was dissolved in EtOAc (100 mL), washed with saturated sodium bicarbonate (1×30 mL) and brine (1×30 mL), dried over sodium sulfate, filtered and concentrated to afford the crude product. The crude was chromatographed (40 g of silica gel, 10% MeOH in dichloromethane) to yield N-{1-[3-(3,5-difluorophenyl)quinoxalin-2-yl]ethyl}-9H-purin-6-amine. MS (ESI+) for C₂₁H₁₅F₂N₇ m/z 404.1 (M+H)⁺. ¹H NMR (CD₃OD) δ 8.15 (m, 4H), 7.928 (s, 1H), 7.87 (m, 2H), 7.45 (d, J=6 Hz, 2H), 7.108 (m, 1H), 5.957 (m, 1H), 1.647 (d, J=6 Hz, 3H).

### Example 2

### Preparation of Compound Q6

### 9-[1-(5-methyl-3-phenylquinoxalin-2-yl)ethyl-]-9H-purin-6-amine

This representative example describes the synthesis of compound Q6, 9-[1-(5-methyl-3-phenylquinoxalin-2-yl)ethyl]-9H-purin-6-amine. The quinoxaline group of this compound is linked to and attached to the 9-position of a purinyl group via a methyl substituted alkylene linker. Shown below is a synthetic scheme followed by a detailed description of each step.

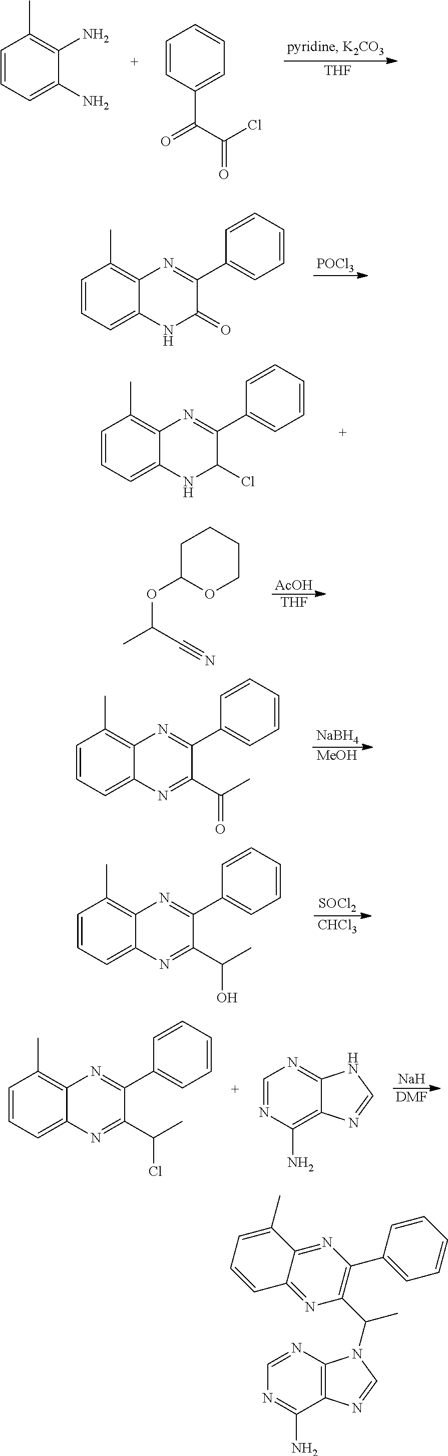

### Step 1: Synthesis of 5-methyl-3-phenylquinoxalin-2(1H)-one

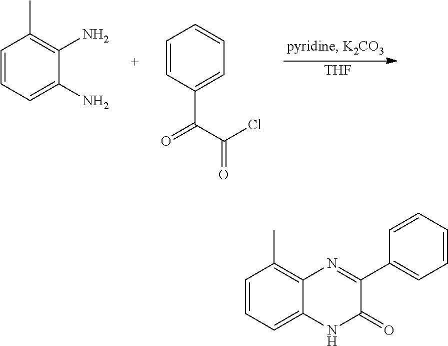

To 3-methylbenzene-1,2-diamine (1.2 g, 9.82 mmol) dissolved in dry dichloromethane (50 mL) was added pyridine (1.19 mL, 14.7 mmol) and potassium carbonate (2.04 g, 14.7 mmol). This was stirred at 21° C. followed by the dropwise addition of benzoylformyl chloride (0.828 g, 4.91 mmol) in dry THF (5 mL). The solution was stirred for 72 hours at 21° C., then diluted with dichloromethane (100 mL) and washed with water (1×50 mL) and brine (1×50 mL). The mixture was dried over sodium sulfate, filtered then evaporated to afford the crude material. This crude material was flash chromatographed (90 g silica gel, 10-30% EtOAc in hexanes) to afford a major fraction and 0.152 g of a less polar minor fraction as white crystals. The major isomer fraction material was concentrated and rechromatographed (90 g silica gel, 5% acetone in dichloromethane) to afford the purified desired product. MS (ESI+) for C₁₅H₁₂N₂O m/z 238.3 (M+H)⁺. ¹H NMR (CDCl₃) δ 11.46 (s, 1H), 8.46 (m, 2H), 7.45 (m, 3H), 7.35 (t, J=8 Hz, 1H), 7.13 (m, 2H), 2.70 (s, 3 H).

### Step 2: Synthesis of 2-chloro-5-methyl-3-phenylquinoxaline

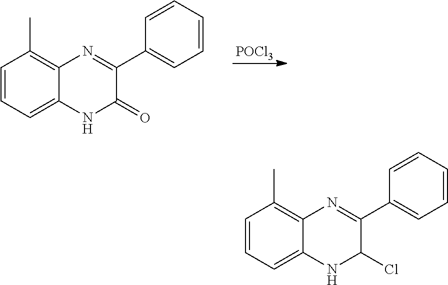

5-Methyl-3-phenylquinoxalin-2(1H)-one (0.9 g, 0.4 mmol) was dissolved in phosphoryl chloride (3.0 mL, 32 mmol) and gently warmed to 100° C. The solution was stirred for 1 hr until the reaction was complete as observed by HPLC. The mixture was cooled and concentrated. The residue was crystallized to form white crystals which were dissolved in ether (100 mL), washed with saturated sodium bicarbonate (1×25 mL) and water (1×25 mL), then dried over sodium sulfate, filtered and evaporated to form the desired product 2-chloro-5-methyl-3-phenylquinoxaline. This was used without further purification. MS (ESI+) for C₁₅H₁₁ClN₂ m/z 255.2 (M+H)⁺. ¹H NMR (CDCl₃) δ 8.11 (m, 1H), 7.93 (m, 3H), 7.83 (m, 1H), 7.63 (m, 3H), 2.91 (s, 3H).

### Step 3: Synthesis of 1-(5-methyl-3-phenylquinoxalin-2-yl)ethanone

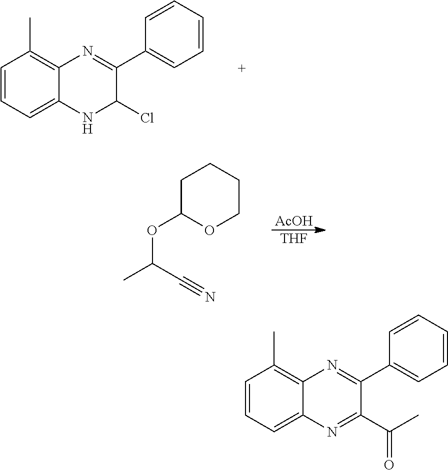 [SMILES: "AcOH" ; Cc1cccc2c1N=C(c1ccccc1)C(Cl)N2 ; CC(C#N)OC1CCCCO1 ; CC(=O)c1nc2cccc(C)c2nc1-c1ccccc1 ; "THF"]

To a 100 mL, flame-dried round-bottomed flask was added 2-(tetrahydro-2H-pyran-2-yloxy)propanenitrile (0.11 g, 0.707 mmol) and 2-chloro-5-methyl-3-phenylquinoxaline (0.090 g, 0.353 mmol) dissolved in dry THF (10 mL). This solution was cooled to −78° C., then LDA (2.0 M solution in THF, 350 μL) was added and the reaction was stirred for 1 hr until all of the starting material was gone (as evidenced by TLC/HPLC). The reaction mixture was allowed to warm to 0° C. then quenched with glacial acetic acid (40.2 μL, 0.707 mmol). The reaction was warmed to 21° C. then the solvent was evaporated. The residue was dissolved in methanol (10 mL), then 5% sulfuric acid (3 mL) was added and the solution was stirred at 21° C. for 18 hours. The solvent was evaporated and the residue was dissolved in ether (20 mL) to which was added 1N KOH until the pH turns basic. This was stirred at 21° C. for 2 hours. The residue was diluted with ether (50 mL), washed with brine (20 mL), dried over sodium sulfate, filtered, evaporated and chromatographed (40 g silica gel, 1-3% EtOAc in hexanes) to give 1-(5-methyl-3-phenylquinoxalin-2-yl)ethanone. MS (ESI+) for C₁₇H₁₄N₂O m/z 263.2 (M+H)⁺. ¹H NMR (CDCl₃) δ 7.93 (m, 1H), 7.63 (m, 4H), 7.42 (m, 3H), 2.77 (s, 3H), 2.69 (s, 3H).

### Step 4: Synthesis of 1-(5-methyl-3-phenylquinoxalin-2-yl)ethanol

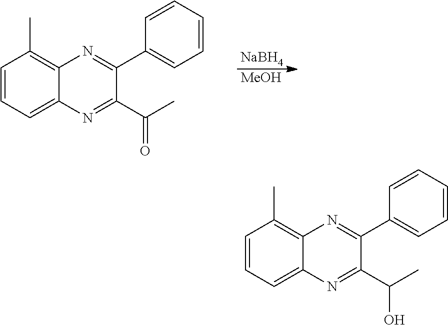

To 1-(5-methyl-3-phenylquinoxalin-2-yl)ethanone (0.180 g, 0.69 mmol) dissolved in methanol (5 mL) and cooled to 0° C., was added sodium borohydride (0.0286 g, 0.755 mmol). This solution was stirred at 0° C. for 1 hour, then warmed to 21° C. and was stirred for 1 hour more. All of the starting material slowly goes into solution with gas evolution. The solvent was evaporated and the residue was dissolved in ether (100 mL), followed by washing with water (1×20 mL) and brine (1×20 mL). This mixture was dried over sodium sulfate, filtered through celite and concentrated by rotary evaporation to yield crude 1-(5-methyl-3-phenylquinoxalin-2-yl)ethanol which was used in the next reaction without further purification. MS (ESI+) for C₁₇H₁₆N₂O m/z 265.3 (M+H)⁺. ¹H NMR (CDCl₃) δ 7.97 (d, J=8 Hz, 1H), 7.70 (m, 3H), 7.63 (m, 1H), 7.56 (m, 3H), 5.45 (s, 1H), 4.78 (s, 1H), 2.83 (s, 3H), 1.25 (d, J=6 Hz, 3H).

### Step 5: Synthesis of 2-(1-chloroethyl)-5-methyl-3-phenylquinoxaline

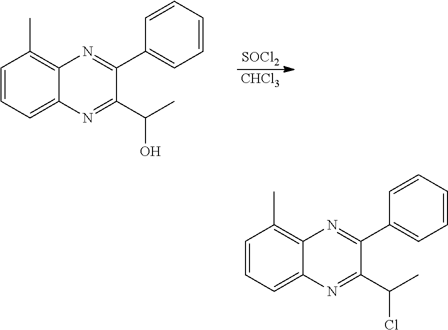

To crude 1-(5-methyl-3-phenylquinoxalin-2-yl)ethanol (0.15 g, 0.567 mmol) dissolved in chloroform (10 mL) was added thionyl chloride (82.8 μL, 1.13 mmol) at 21° C. After 1 hour, pyridine (119 μL, 1.48 mmol) was added and the reaction was warmed to 50° C. and stirred for 2 hours. The solvent was evaporated and the residue was dissolved in EtOAc (100 mL), washed with water (1×25 mL), saturated sodium bicarbonate (1×25 mL) and brine (1×25 mL). This was dried over sodium sulfate, treated with decolorizing carbon, filtered, then concentrated by rotary evaporation to give the crude product (0.160 g) as a pale red oil which slowly crystallized. The crude chloride was chromatographed (silica gel, 5% EtOAc in hexanes) to give 2-(1-chloroethyl)-5-methyl-3-phenylquinoxaline as a white solid. MS (ESI+) for C₁₇H₁₅ClN₂ m/z 283.2 (M+H)⁺. ¹H NMR (CDCl₃) δ 8.04 (d, J=8 Hz, 1H), 7.81 (m, 2H), 7.70 (m, 1H), 7.64 (m, 1H), 7.58 (m, 3H), 5.52 (q, J=6 Hz, 1H), 2.82 (s, 3H), 2.01 (d, J=6 Hz, 3H).

### Step 6: Synthesis of 9-[1-(5-methyl-3-phenylquinoxalin-2-yl)ethyl]-9H-purin-6-amine

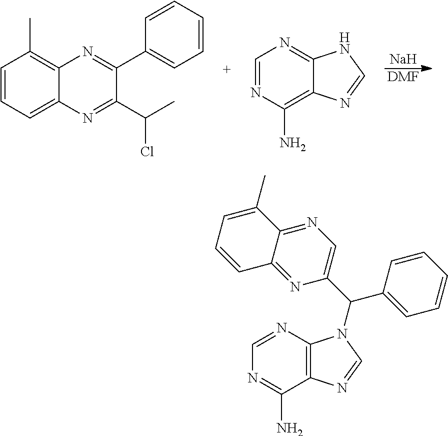

To a flame-dried, 25 mL round-bottomed flask under nitrogen was added adenine (0.059 g, 0.44 mmol) dissolved in DMF. To this was added sodium hydride (60% in mineral oil) (0.0276 g, 0.690 mmol). The reaction mixture was warmed to 75° C. in an oil bath until gas evolution ceases and the mixture becomes pasty and blue-grey in color, (about 30 minutes). A solution of 2-(1-chloroethyl)-5-methyl-3-phenylquinoxaline dissolved in dry DMF (5 mL) was added to the mixture at 75° C. and stirring was continued for 1 hour. The DMF was evaporated under a stream of dry nitrogen and the resulting residue was dissolved in EtOAc (150 mL). This was quenched with saturated ammonium chloride (15 mL), diluted with water (20 mL) and extracted. The organic layer was washed with water (1×20 mL) and brine (1×20 mL), dried over sodium sulfate, filtered and concentrated by rotary evaporation to give the crude product. The crude was chromatographed (40 g of silica gel, 1-5% methanol in EtOAc) to give 0.058 g of an off-white solid (94% purity by HPLC). This residue was triturated with ether and dried on hi-vac for 18 hours to give the title product 9-[1-(5-methyl-3-phenylquinoxalin-2-yl)ethyl]-9H-purin-6-amine as a clean white solid. MS (ESI+) for C₂₂H₁₉N₇ m/z 382.2 (M+H)⁺. ¹H NMR (CDCl₃) δ 8.31 (d, J=20 Hz, 2H), 7.94 (d, J=10 Hz, 1H), 7.74 (m, 2H), 7.68 (m, 1H), 7.63 (m, 1H), 7.55 (m, 3H), 6.58 (q, J=7 Hz, 1H), 5.50 (s, 2H), 2.80 (s, 3H), 1.88 (d, J=7 Hz, 3H).

### Example 3

### PI3K Biochemical Enzyme Assay

This example describes a method of obtaining in vitro activity data concerning the effect of compounds of on various PI3K isoforms. The effect of compounds on the kinase activity of Class I PI3Ks was measured at Invitrogen by using the Adapta® universal kinase assay. It was a homogenous, fluorescent based immunoassay for the detection of ADP produced by the kinase reaction. This assay can be divided into two phases: a kinase reaction phase, and an ADP detection phase. In the kinase reaction phase, all components required for the kinase reaction were added to the well, and the reaction was allowed to incubate for 60 minutes. After the reaction, a detection solution consisting of a europium labeled anti-ADP antibody, an Alexa Fluor® 647 labeled ADP tracer, and EDTA (to stop the kinase reaction) was added to the assay well. ADP formed by the kinase reaction (in the absence of an inhibitor) displaced the Alexa Fluor® 647 labeled ADP tracer from the antibody, resulting in a decrease in the TR-FRET signal. In the presence of an inhibitor, the amount of ADP formed by the kinase reaction was reduced, and the resulting intact antibody-tracer interaction resulted in a high TR-FRET signal.

ADP formation was determined by calculating the emission ratio from the assay well. The emission ratio was calculated by dividing the intensity of the tracer (acceptor) emission by the intensity of the Eu (donor) emission at 615 nm as shown in the equation below.

$$Emission Ratio = \frac{{AlexaFluor}^{®} 647 Emission ( 665 nm )}{Europium Emission ( 615 nm )}$$

The Test Compounds were screened in 1% DMSO (final) in the well. All Substrate/Kinase Mixtures were diluted to a 2× working concentration in the appropriate Kinase Buffer as described below for the individual kinase:

p110 Alpha/p85 Alpha

The 2× p110 alpha/p85 alpha/PIP2:PS mixture was prepared in 50 mM HEPES pH 7.5, 100 mM NaCl, 0.03% CHAPS, 3 mM mgCl₂, 1 mM EGTA. The final 10 μL Kinase Reaction consisted of 0.3-1.5 ng p110 alpha/p85 alpha and 50 μM PIP2:PS in 32.5 mM HEPES pH 7.5, 50 mM NaCl, 0.015% CHAPS, 1.5 mM mgCl₂, 0.5 mM EGTA. After the 1 hour Kinase Reaction incubation, 5 μL of Detection Mix was added.

p110 Beta/p85 Alpha

The 2× p 110beta/p85 alpha/PIP2:PS mixture was prepared in 50 mM HEPES pH 7.5, 100 mM NaCl, 0.03% CHAPS, 1 mM EGTA, 3 mM mgCl₂, and 2 mM DTT. The final 10 μL Kinase Reaction consisted of 35.4 ng p110 beta/p85 alpha and 50 μM PIP2:PS. After the 1 hour Kinase Reaction incubation, 5 μL of Detection Mix was added.

p110 Delta/p85 Alpha

The 2× p110 delta/p85 alpha/PIP2:PS mixture was prepared in 50 mM HEPES pH 7.5, 100 mM NaCl, 0.03% CHAPS, 3 mM mgCl₂, 1 mM EGTA. The final 10 μL Kinase Reaction consisted of 0.35-2.6 ng p110 delta/p85 alpha and 50 μM PIP2:PS in 32.5 mM HEPES pH 7.5, 50 mM NaCl, 0.015% CHAPS, 1.5 mM mgCl₂, 0.5 mM EGTA. After the 1 hour Kinase Reaction incubation, 50 μL of Detection Mix was added.

p110 Gamma

The 2× p110 gamma/PIP2:PS mixture was prepared in 50 mM HEPES pH 7.5, 1 mM EGTA, 3 mM mgCl₂. The final 100 μL Kinase Reaction consisted of 3.5-26 ng p110 gamma and 50 μM PIP2:PS in 32.5 mM HEPES pH 7.5, 0.5 mM EGTA, 1.5 mM mgCl₂. After the 1 hour Kinase Reaction incubation, 5 μL of Detection Mix was added.

The Detection mix consisted of EDTA (30 mM), Eu-anti-ADP antibody (30 nM) and ADP tracer. All ATP Solutions were diluted to a 4× working concentration in water and used at the Km apparent concentrations for each individual kinase.

### Example 4

### PI3K Isoform Activity of Compounds

This example provides in vitro activity data of compounds Q1-Q17 for various PI3K isoforms. The structure of compounds Q1-Q17 is shown in the previous sections of the specification. Data gathered in Table 1 may be obtained using the methods described in previous example and reflects the percent inhibition at a certain concentration (i.e., at 10, 1, 0.1 μM). The table gives insight into the activity of the compounds in inhibiting PI3Kα, β, γ and δ activity. The compounds are generally selective for PI3Kδ, and to some extent PI3Kγ, relative to PI3Kα and PI3Kβ. Table 2 below summarizes examples of quinoxaline derivatives and their IC₅₀ values in PI3k biochemical assays.

TABLE 1

| | PI3Kα | PI3Kβ | PI3Kδ | PI3Kγ |
|---|---|---|---|---|
| | % inhib | % inhib | % inhib | % inhib |
| | 10, 1, | 10, 1, | 10, 1, | 10, 1, |
| Compound | 0.1 μM | 0.1 μM | 0.1 μM | 0.1 μM |
| Q1 | 89, 18, — | 72, —, — | 100, 85, 74 | 99, 78, — |
| Q2 | 47, —, — | 8, —, — | 100, 82, 20 | 86, 42, — |
| Q3 | 65, —, — | 25, —, — | 100, 86, 48 | 94, 51, — |
| Q4 | 92, 18, — | 77, —, — | 100, 86, 77 | 99, 84, 36 |
| Q5* | 97, 41, — | 85, 23, — | 99, 69, — | 98, 79, — |
| Q6 | 32, —, — | 61, —, — | 100, 86, 63 | 89, 48, — |
| Q7* | 88, 28, — | 77, —, — | 100, 71, — | 95, 61, — |
| Q8 | 50, —, — | 53, —, — | 100, 58, — | 83, 30, — |
| Q9 | 72, —, — | 60, —, — | 100, 86, 72 | 95, 37, — |
| Q10 | 30, —, — | 30, —, — | 99, 74, — | 82, 12, — |
| Q11 | 53, —, — | 76, —, — | 99, 45, — | 79, —, — |
| Q12 | 77, —, — | 75, —, — | 100, 83, 71 | 96, 44, — |

| | PI3Kα | PI3Kβ | PI3Kδ | PI3Kγ |
|---|---|---|---|---|
| Compound | IC50 (nM) | IC50 (nM) | IC50 (nM) | IC50 (nM) |
| Q13 | — | — | 1,175 | 46,500 |
| Q14 | — | — | 950 | 17,500 |
| Q15 | — | — | 250 | 2,375 |
| Q16 | 51,000 | 14,500 | 190 | 20,500 |
| Q17 | 100,000 | 8,500 | 75 | 692 |

*HLM 1 μM % remaining, 3A4 10 μM % remaining: Q5 = 85%, 89% (AP); Q7 = 85%, −5%

TABLE 2

| Compound | p110β/p85α | p120γ | p110δ/p85α | p110α/p85α |
|---|---|---|---|---|
| Q1 | >10,000 | 147 | 14 | 6,917 |
| Q2 | >10,000 | 5,785 | 58 | >10,000 |
| Q3 | >10,000 | 2,072 | 36 | >10,000 |
| Q4 | 1,126 | 8 | 2 | 2,593 |
| Q5 | 459 | 2 | 2 | 404 |
| Q9 | >10,000 | 2,145 | 37 | >10,000 |
| Q12 | >10,000 | 762 | 17 | >10,000 |

### Example 5

### PI3K Isoform Specific Cell-Based Assays

PDGF-BB-induced AKT phosphorylation in Swiss-3T3 fibroblasts is mediated by p110α. C5a-induced AKT phosphorylation in RAW-264 murine macrophages is mediated exclusively by p110γ. To assess the activity of compounds on inhibition of these isoform in cells, we treated Swiss 3T3 or RAW-264 cells with either vehicle or serial dilutions of compounds prior to agonist-treatment and measured the level of AKT phosphorylation as described below.

PI3Kα-Dependent Cell-Based Assay: PDGF-BB Mediated AKT Phosphorylation in Swiss-3T3 Cells

Swiss-3T3 fibroblasts (American Type Culture Collection) were cultured with DMEM containing 10% fetal bovine serum and the antibiotics penicillin and streptomycin. Cells were seeded on a 96-well Tissue Culture Plate at 25,000 cells/well and allowed to reach at least 90% confluency. The cells were starved for 2 to 12 hr in 0.1% FBS containing medium and then were pretreated with inhibitors or DMSO for 2 hr. The cells were stimulated for 15 min with 10 ng/ml PDGF-BB (Cell Signaling Technology) at 37° C. in 5% CO₂. The culture medium was removed and cells were fixed for 20 min at room temperature by addition of 100 μl of 4% cell fixing buffer to each well. AKT phosphorylation and total AKT was detected by ELISA.

PI3Kγ-Dependent Cell-Based Assay: C5a-Mediated AKT Phosphorylation in RAW-264 Cells

RAW-264 macrophage cells (American Type Culture Collection) were cultured with Dulbecco's modified Eagle medium (DMEM) containing 10% fetal bovine serum and the antibiotics penicillin and streptomycin. Cells were seeded on a 96-well Tissue Culture Plate at 100,000 to 200,000 cells/well the day before the experiment. Next day, cells were starved for 2 hr in 0.1% FBS containing medium. The cells were pretreated with inhibitors or DMSO for 2 hr and stimulated for 5 min with 75 ng/ml C5a (R&D) at 37° C. in 5% CO₂. The culture medium was removed and cells were fixed for 20 min at room temperature by addition of 100 μl of 4% cell fixing buffer to each well. AKT phosphorylation and total AKT was detected by ELISA.

Detection of Phospho-Ser-473 AKT and Total AKT by ELISA

The fixed cells were washed twice with 150 μl of wash buffer (WB) and quenched by incubating with 100 μl of Quenching Buffer for 20 min at room temperature. Cells were washed once with 150 μl WB and blocked by incubating with 100 μl of Blocking Buffer for 1 hr at room temperature. Cells were incubated with 50 μl of primary antibody diluted in Blocking Buffer, either phospho-Ser-473 AKT specific (1:150 dilution) or total-AKT antibody (1:200 dilution), to each specific well. The negative control wells contained 50 μl of Blocking Buffer. Plates were sealed with plate sealing film and incubated overnight at 4° C. Cells were washed twice with 150 μl WB followed by addition of 50 μl per well of DELFIA secondary antibody (50 ng/well, diluted in DELFIA Assay Buffer). After 2 hr incubation at room temperature, cells were washed 4 times with 150 μl of Europium Wash Solution followed by addition of 100 μl of DELFIA Enhancing Solution to each well. Plates were incubated for 5 min in the dark and fluorescent signal was read using SpectraMax M5™ (Molecular Devices). Total AKT level was determined to verify equal cell number per well. The average background fluorescence values from wells that received vehicle control in the absence of PDGF-BB or C5a were subtracted from all the values of experimental conditions. Fluorescence values corresponding to phosphorylated AKT from wells that received PDGF-BB or C5a were normalized to 100% and compound effect was plotted as % change relative to the vehicle control.

TABLE 1

| Compound | PI3Kα EC50 (nM) | PI3Kγ EC50 (nM) |
|---|---|---|
| Q1 | >20,000 | 4,623 |
| Q2 | >20,000 | >20,000 |
| Q3 | >20,000 | >20,000 |
| Q4 | 17,702 | 344 |
| Q5* | 14,329 | 628 |
| Q6 | >20,000 | >20,000 |
| Q7* | 15,245 | 3,588 |
| Q8 | >20,000 | >20,000 |
| Q9 | >20,000 | >20,000 |
| Q10 | >20,000 | >20,000 |
| Q11 | >20,000 | >20,000 |
| Q12 | >20,000 | >20,000 |

*HLM 1 μM % remaining, 3A4 10 μM % remaining: Q5 = 85%, 89% (AP); Q7 = 85%, −5%

1. A compound of Formula I or a pharmaceutically acceptable salt thereof, 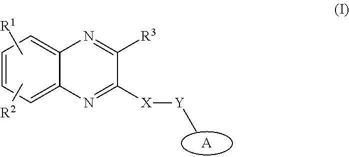
wherein A is a monocyclic or bicyclic ring system containing at least two nitrogen atoms, and at least one ring of the system is aromatic;
wherein A is optionally substituted with 1-3 substituents;
X is selected from the group consisting of C(R^{b})₂, CH₂CHR^{b}, and CH═C(R^{b});
Y is selected from the group consisting of null, S, SO, SO₂, NR^{d}, O, C(═O), OC(═O), C(═O)O, and NHC(═O)CH₂S;
R¹ and R², independently, are selected from the group consisting of hydrogen, halo, NO₂, CF₃, OCF₃, and CN, or from the group consisting of C₁₋₆alkyl, aryl, heteroaryl, NHC(═O)C₁₋₃alkyleneN(R^{a})₂, OR^{a}, N(R^{a})₂, OC(═O)R^{a}, C(═O)R^{a}, C(═O)OR^{a}, arylOR^{a}, Het, NR^{a}C(═O)C₁₋₃alkyleneC(═O)OR^{a}, arylOC₁₋₃alkyleneN(R^{a})₂, arylOC(═O)R^{a}, C₁₋₄alkyleneC(═O)OR^{a}, OC₁₋₄alkyleneC(═O)OR^{a}, C₁₋₄alkyleneOC₁₋₄alkyleneC(═O)OR^{a}, C(═O)NR^{a}SO₂R^{a}, C₁₋₄alkyleneN(R^{a})₂, C₂₋₆alkenyleneN(R^{a})₂, C(═O)NR^{a}C₁₋₄alkyleneOR^{a}, C(═O)NR^{a}C₁₋₄alkyleneHet, OC₂₋₄alkyleneN(R^{a})₂, OC₁₋₄alkyleneCH(OR^{a})CH₂N(R^{a})₂, OC₁₋₄alkyleneHet, OC₂₋₄alkyleneOR^{a}, OC₂₋₄alkyleneNR^{a}C(═O)OR^{a}, NR^{a}C₁₋₄alkyleneN(R^{a})₂, NR^{a}C(═O)R^{a}, NR^{a}C(═O)N(R^{a})₂, N(SO₂C₁₋₄alkyl)₂, NR^{a}(SO₂C₁₋₄alkyl), SO₂N(R^{a})₂, OSO₂CF₃, C₁₋₃alkylenearyl, C₁₋₄alkyleneHet, C₁₋₆alkyleneOR^{a}, C₁₋₃alkyleneN(R^{a})₂, C(═O)N(R^{a})₂, NHC(═O)C₁₋₃alkylenearyl, C₃₋₈cycloalkyl, C₃₋₈heterocycloalkyl, arylOC₁₋₃alkyleneN(R^{a})₂, arylOC(═O)R^{a}, NHC(═O)C₁₋₃alkyleneC₃₋₈heterocycloalkyl, NHC(═O)C₁₋₃alkyleneHet, OC₁₋₄alkyleneOC₁₋₄alkyleneC(═O)OR^{a}, C(═O)C₁₋₄alkyleneHet, and NHC(═O)haloC₁₋₆alkyl, each of which is optionally substituted;
or R¹ and R² are taken together to form a 3- or 4-membered alkylene or alkenylene chain component of a 5- or 6-membered ring, optionally containing at least one heteroatom selected from the group consisting of N, O, and S;
R³ is hydrogen or is a member selected from the group consisting of C₁₋₆alkyl, C₃₋₈cycloalkyl, C₃₋₈heterocycloalkyl, C₁₋₄alkylenecycloalkyl, C₂₋₆alkenyl, C₁₋₃alkylenearyl, arylC₁₋₃alkyl, C(═O)R^{a}, aryl, heteroaryl, C(═O)OR^{a}, C(═O)N(R^{a})₂, C(═S)N(R^{a})₂, SO₂R^{a}, SO₂N(R^{a})₂, S(═O)R^{a}, S(═O)N(R^{a})₂, C(═O)NR^{a}C₁₋₄alkyleneOR^{a}, C(═O)NR^{a}C₁₋₄alkyleneHet, C(═O)C₁₋₄alkylenearyl, C(═O)C₁₋₄alkyleneheteroaryl, and C₁₋₄alkylenearyl, each of which is optionally substituted with 1-3 substitutents;
each R^{a} is independently selected from hydrogen or from the group consisting of C₁₋₆alkyl, C₃₋₈cycloalkyl, C₃₋₈heterocycloalkyl, C₁₋₃alkyleneN(R^{c})₂, aryl, arylC₁₋₃alkyl, C₁₋₃alkylenearyl, heteroaryl, heteroarylC₁₋₃alkyl, and C₁₋₃alkyleneheteroaryl, each of which is optionally substituted;
or two R^{a} groups on the same atom or on adjacent atoms are taken together to form a 5- or 6-membered ring, optionally containing at least one heteroatom;
each R^{b} is independently selected from the group consisting of hydrogen, halo and CN or from the group consisting of C₁₋₆alkyl, C₁₋₆haloalkyl, C(═O)R^{a}, C(═O)OR^{a}, heteroC₁₋₃alkyl, C₁₋₃alkyleneheteroC₁₋₃alkyl, arylheteroC₁₋₃alkyl, aryl, heteroaryl, arylC₁₋₃alkyl, heteroarylC₁₋₃alkyl, C₁₋₃alkylenearyl, and C₁₋₃alkyleneheteroaryl, each of which is optionally substituted; or R^{b} and R^{d} can be taken together to form a 5-7 membered optionally substituted ring;
each R^{c} is independently selected from hydrogen or from the group consisting of C₁₋₆alkyl, C₃₋₈cycloalkyl, aryl, and heteroaryl, each of which is optionally substituted;
wherein R^{d} is H or C₁₋₁₀acyl; or R^{d} and R^{b}, if X comprises R^{b}, can be taken together to form a 5-7 membered optionally substituted ring; and
each Het is a 5- or 6-membered heterocyclic ring, wherein said heterocyclic ring is saturated, partially unsaturated or aromatic, and said heterocyclic ring contains at least one heteroatom selected from the group consisting of N, O, and S; wherein Het is optionally substituted with 1-3 substituents.

2. The compound according to claim 1, wherein X is C(R^{b})₂ or CH₂CHR^{b}; and wherein X has a chiral center.

3. The compound according to claim 2, wherein the chiral center is the S-enantiomer.

4. The compound according to claim 1, wherein A is selected from the group consisting of 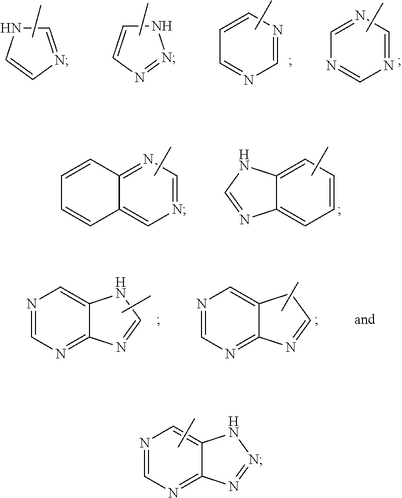
each of which is optionally substituted.

5. The compound according to claim 4, wherein A is a purinyl ring.

6. The compound according to claim 4, wherein A is optionally substituted with 1-3 substituents independently selected from the group consisting of N(R^{a})₂, halo, CN, C₁₋₆alkyl, C₁₋₆haloalkyl C(═O)R^{a}, and C(═O)OR^{a}.

7. The compound according to claim 1, wherein R³ is optionally substituted aryl.

8. The compound according to claim 7, wherein R³ is phenyl optionally substituted with 1-3 substituents independently selected from the group consisting of N(R^{a})₂, halo, CN, C₁₋₆alkyl, OR^{a}, C₁₋₆halo alkyl C(═O)R^{a}, and C(═O)OR^{a}.

9. The compound according to claim 1, wherein X is CH(R^{b}).

10. The compound according to claim 9, wherein X is selected from the group consisting of CH₂, CH(CH₂)₀₋₂CH₃, CHCH(CH₃)₂, C(CH₃)₂, and CHCH((CH₂)₀₋₁CH₃)₂, each of which is optionally substituted.

11. The compound according to claim 1, wherein Y is NH or S.

12. The compound according to claim 1 or a pharmaceutically acceptable salt thereof, wherein the compound is represented by Formula II 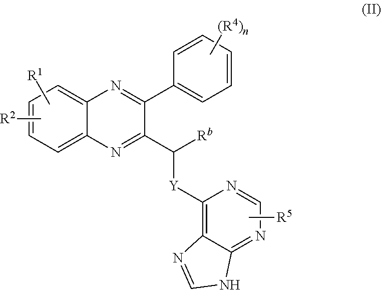
wherein each R⁴ is independently selected from the group consisting of hydrogen, halo, NO₂, CF₃, OCF₃, and CN, or from the group consisting of C₁₋₆alkyl, aryl, heteroaryl, NHC(═O)C₁₋₃alkyleneN(R^{a})₂, OR^{a}, N(R^{a})₂, OC(═O)R^{a}, C(═O)R^{a}, C(═O)OR^{a}, arylOR^{a}, Het, NR^{a}C(═O)C₁₋₃alkyleneC(═O)OR^{a}, arylOC₁₋₃alkyleneN(R^{a})₂, arylOC(═O)R^{a}, C₁₋₄alkyleneC(═O)OR^{a}, OC₁₋₄alkyleneC(═O)OR^{a}, C₁₋₄alkyleneOC₁₋₄alkyleneC(═O)OR^{a}, C(═O)NR^{a}SO₂R^{a}, C₁₋₄alkyleneN(R^{a})₂, C₂₋₆alkenyleneN(R^{a})₂, C(═O)NR^{a}C₁₋₄alkyleneOR^{a}, C(═O)NR^{a}C₁₋₄alkyleneHet, OC₂₋₄alkyleneN(R^{a})₂, OC₁₋₄alkyleneCH(OR^{a})CH₂N(R^{a})₂, OC₁₋₄alkyleneHet, OC₂₋₄alkyleneOR^{a}, OC₂₋₄alkyleneNR^{a}C(═O)OR^{a}, NR^{a}C₁₋₄alkyleneN(R^{a})₂, NR^{a}C(═O)R^{a}, NR^{a}C(═O)N(R^{a})₂, N(SO₂C₁₋₄alkyl)₂, NR^{a}(SO₂C₁₋₄alkyl), SO₂N(R^{a})₂, OSO₂CF₃, C₁₋₃alkylenearyl, C₁₋₄alkyleneHet, C₁₋₆alkyleneOR^{a}, C₁₋₃alkyleneN(R^{a})₂, C(═O)N(R^{a})₂, NHC(═O)C₁₋₃alkylenearyl, C₃₋₈cycloalkyl, C₃₋₈heterocycloalkyl, arylOC₁₋₃alkyleneN(R^{a})₂, arylOC(═O)R^{a}, NHC(═O)C₁₋₃alkyleneC₃₋₈heterocycloalkyl, NHC(═O)C₁₋₃alkyleneHet, OC₁₋₄alkyleneOC₁₋₄alkyleneC(═O)OR^{a}, C(═O)C₁₋₄alkyleneHet, and NHC(═O)haloC₁₋₆alkyl, each of which is optionally substituted;
or two R⁴ groups are taken together to form a 3- or 4-membered alkylene or alkenylene chain component of a 5- or 6-membered ring, optionally containing at least one heteroatom selected from the group consisting of N, O, and S;
n is 0-3; and
R⁵ is selected from the group consisting of hydrogen, halo, NH₂, NO₂, CF₃, OCF₃, and CN, or from the group consisting of C₁₋₆alkyl, aryl, heteroaryl, NHC(═O)C₁₋₃alkyleneN(R^{a})₂, OR^{a}, N(R^{a})₂, OC(═O)R^{a}, C(═O)R^{a}, C(═O)OR^{a}, arylOR^{a}, Het, NR^{a}C(═O)C₁₋₃alkyleneC(═O)OR^{a}, arylOC₁₋₃alkyleneN(R^{a})₂, arylOC(═O)R^{a}, C₁₋₄alkyleneC(═O)OR^{a}, OC₁₋₄alkyleneC(═O)OR^{a}, C₁₋₄alkyleneOC₁₋₄alkyleneC(═O)OR^{a}, C(═O)NR^{a}SO₂R^{a}, C₁₋₄alkyleneN(R^{a})₂, C₂₋₆alkenyleneN(R^{a})₂, C(═O)NR^{a}C₁₋₄alkyleneOR^{a}, C(═O)NR^{a}C₁₋₄alkyleneHet, OC₂₋₄alkyleneN(R^{a})₂, OC₁₋₄alkyleneCH(OR^{a})CH₂N(R^{a})₂, OC₁₋₄alkyleneHet, OC₂₋₄alkyleneOR^{a}, OC₂₋₄alkyleneNR^{a}C(═O)OR^{a}, NR^{a}C₁₋₄alkyleneN(R^{a})₂, NR^{a}C(═O)R^{a}, NR^{a}C(═O)N(R^{a})₂, N(SO₂C₁₋₄alkyl)₂, NR^{a}(SO₂C₁₋₄alkyl), SO₂N(R^{a})₂, OSO₂CF₃, C₁₋₃alkylenearyl, C₁₋₄alkyleneHet, C₁₋₆alkyleneOR^{a}, C₁₋₃alkyleneN(R^{a})₂, C(═O)N(R^{a})₂, NHC(═O)C₁₋₃alkylenearyl, C₃₋₈cycloalkyl, C₃₋₈heterocycloalkyl, arylOC₁₋₃alkyleneN(R^{a})₂, arylOC(═O)R^{a}, NHC(═O)C₁₋₃alkyleneC₃₋₈heterocycloalkyl, NHC(═O)C₁₋₃alkyleneHet, OC₁₋₄alkyleneOC₁₋₄alkyleneC(═O)OR^{a}, C(═O)C₁₋₄alkyleneHet, and NHC(═O)haloC₁₋₆alkyl, each of which is optionally substituted.

13. The compound according to claim 12, wherein
R¹ and R², independently, are selected from the group consisting of hydrogen, F, Cl, Br, NO₂, CF₃, OCF₃, and CN, or from the group consisting of methyl, ethyl, propyl, butyl, phenyl, heteroaryl, OR^{a}, N(R^{a})₂, OC(═O)R^{a}, C(═O)R^{a}, C(═O)OR^{a}, each of which is optionally substituted;
R^{b} is selected from the group consisting of hydrogen, halo, and CN or from the group consisting of methyl, ethyl, propyl, butyl, C(═O)R^{a}, and C(═O)OR^{a}, each of which may be optionally substituted;
each R⁴ is independently selected from the group consisting of hydrogen, F, Cl, Br, NO₂, CN, CF₃, and OCF₃, or from the group consisting of methyl, ethyl, propyl, butyl, phenyl, heteroaryl, OR^{a}, N(R^{a})₂, OC(═O)R^{a}, C(═O)R^{a}, C(═O)OR^{a}, Het, each of which is optionally substituted;
n is 0-2; and
R⁵ is selected from the group consisting of hydrogen, F, Cl, Br, NH₂, NO₂, CN, CF₃, and OCF₃, or from the group consisting of methyl, ethyl, propyl, butyl, phenyl, heteroaryl, OR^{a}, N(R^{a})₂, OC(═O)R^{a}, C(═O)R^{a}, C(═O)OR^{a}, Het, each of which is optionally substituted.

14. The compound according to claim 1 or a pharmaceutically acceptable salt thereof, wherein the compound has a Formula III 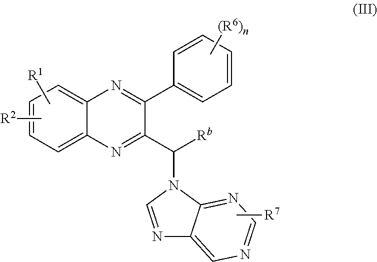
wherein R^{b} is selected from the group consisting of hydrogen, halo, and CN or from the group consisting of C₁₋₆alkyl, C(═O)R^{a}, and C(═O)OR^{a}, each of which may be optionally substituted;
each R⁶ is independently selected from the group consisting of hydrogen, halo, NO₂, CF₃, OCF₃, and CN, or from the group consisting of C₁₋₆alkyl, aryl, heteroaryl, NHC(═O)C₁₋₃alkyleneN(R^{a})₂, OR^{a}, N(R^{a})₂, OC(═O)R^{a}, C(═O)R^{a}, C(═O)OR^{a}, arylOR^{a}, Het, NR^{a}C(═O)C₁₋₃alkyleneC(═O)OR^{a}, arylOC₁₋₃alkyleneN(R^{a})₂, arylOC(═O)R^{a}, C₁₋₄alkyleneC(═O)OR^{a}, OC₁₋₄alkyleneC(═O)OR^{a}, C₁₋₄alkyleneOC₁₋₄alkyleneC(═O)OR^{a}, C(═O)NR^{a}SO₂R^{a}, C₁₋₄alkyleneN(R^{a})₂, C₂₋₆alkenyleneN(R^{a})₂, C(═O)NR^{a}C₁₋₄alkyleneOR^{a}, C(═O)NR^{a}C₁₋₄alkyleneHet, OC₂₋₄alkyleneN(R^{a})₂, OC₁₋₄alkyleneCH(OR^{a})CH₂N(R^{a})₂, OC₁₋₄alkyleneHet, OC₂₋₄alkyleneOR^{a}, OC₂₋₄alkyleneNR^{a}C(═O)OR^{a}, NR^{a}C₁₋₄alkyleneN(R^{a})₂, NR^{a}C(═O)R^{a}, NR^{a}C(═O)N(R^{a})₂, N(SO₂C₁₋₄alkyl)₂, NR^{a}(SO₂C₁₋₄alkyl), SO₂N(R^{a})₂, OSO₂CF₃, C₁₋₃alkylenearyl, C₁₋₄alkyleneHet, C₁₋₆alkyleneOR^{a}, C₁₋₃alkyleneN(R^{a})₂, C(═O)N(R^{a})₂, NHC(═O)C₁₋₃alkylenearyl, C₃₋₈cycloalkyl, C₃₋₈heterocycloalkyl, arylOC₁₋₃alkyleneN(R^{a})₂, arylOC(═O)R^{a}, NHC(═O)C₁₋₃alkyleneC₃₋₈heterocycloalkyl, NHC(═O)C₁₋₃alkyleneHet, OC₁₋₄alkyleneOC₁₋₄alkyleneC(═O)OR^{a}, C(═O)C₁₋₄alkyleneHet, and NHC(═O)haloC₁₋₆alkyl, each of which is optionally substituted;
or two R⁶ groups are taken together to form a 3- or 4-membered alkylene or alkenylene chain component of a 5- or 6-membered ring, optionally containing at least one heteroatom selected from the group consisting of N, O and S;
n is 0-3; and
R⁷ is selected from the group consisting of hydrogen, halo, NO₂, CF₃, OCF₃, and CN, or from the group consisting of C₁₋₆alkyl, aryl, heteroaryl, NHC(═O)C₁₋₃alkyleneN(R^{a})₂, OR^{a}, N(R^{a})₂, OC(═O)R^{a}, C(═O)R^{a}, C(═O)OR^{a}, arylOR^{a}, Het, NR^{a}C(═O)C₁₋₃alkyleneC(═O)OR^{a}, arylOC₁₋₃alkyleneN(R^{a})₂, arylOC(═O)R^{a}, C₁₋₄alkyleneC(═O)OR^{a}, OC₁₋₄alkyleneC(═O)OR^{a}, C₁₋₄alkyleneOC₁₋₄alkyleneC(═O)OR^{a}, C(═O)NR^{a}SO₂R^{a}, C₁₋₄alkyleneN(R^{a})₂, C₂₋₆alkenyleneN(R^{a})₂, C(═O)NR^{a}C₁₋₄alkyleneOR^{a}, C(═O)NR^{a}C₁₋₄alkyleneHet, OC₂₋₄alkyleneN(R^{a})₂, OC₁₋₄alkyleneCH(OR^{a})CH₂N(R^{a})₂, OC₁₋₄alkyleneHet, OC₂₋₄alkyleneOR^{a}, OC₂₋₄alkyleneNR^{a}C(═O)OR^{a}, NR^{a}C₁₋₄alkyleneN(R^{a})₂, NR^{a}C(═O)R^{a}, NR^{a}C(═O)N(R^{a})₂, N(SO₂C₁₋₄alkyl)₂, NR^{a}(SO₂C₁₋₄alkyl), SO₂N(R^{a})₂, OSO₂CF₃, C₁₋₃alkylenearyl, C₁₋₄alkyleneHet, C₁₋₆alkyleneOR^{a}, C₁₋₃alkyleneN(R^{a})₂, C(═O)N(R^{a})₂, NHC(═O)C₁₋₃alkylenearyl, C₃₋₈cycloalkyl, C₃₋₈heterocycloalkyl, arylOC₁₋₃alkyleneN(R^{a})₂, arylOC(═O)R^{a}, NHC(═O)C₁₋₃alkyleneC₃₋₈heterocycloalkyl, NHC(═O)C₁₋₃alkyleneHet, OC₁₋₄alkyleneOC₁₋₄alkyleneC(═O)OR^{a}, C(═O)C₁₋₄alkyleneHet, and NHC(═O)haloC₁₋₆alkyl, each of which is optionally substituted.

15. The compound according to claim 14,
wherein R¹ and R², independently, are selected from the group consisting of hydrogen, F, Cl, Br, NO₂, CF₃, OCF₃, and CN, or from the group consisting of methyl, ethyl, propyl, butyl, phenyl, heteroaryl, OR^{a}, N(R^{a})₂, OC(═O)R^{a}, C(═O)R^{a}, C(═O)OR^{a}, each of which is optionally substituted;
R^{b} is selected from the group consisting of hydrogen, halo, and CN, or from the group consisting of methyl, ethyl, propyl, butyl, C(═O)R^{a}, and C(═O)OR^{a}, each of which may be optionally substituted;
each R⁶ is independently selected from the group consisting of hydrogen, F, Cl, Br, NO₂, OMe, CN, CF₃, and OCF₃, or from the group consisting of methyl, ethyl, propyl, butyl, phenyl, heteroaryl, OR^{a}, N(R^{a})₂, OC(═O)R^{a}, C(═O)R^{a}, C(═O)OR^{a}, Het, each of which is optionally substituted;
n is 0-2; and
R⁷ is selected from the group consisting of hydrogen, F, Cl, Br, NO₂, CN, CF₃, NH₂, and OCF₃, or from the group consisting of methyl, ethyl, propyl, butyl, phenyl, heteroaryl, OR^{a}, N(R^{a})₂, OC(═O)R^{a}, C(═O)R^{a}, C(═O)OR^{a}, Het, each of which is optionally substituted.

16. The compound according to claim 1, wherein the compound of Formula I is selected from the group consisting of 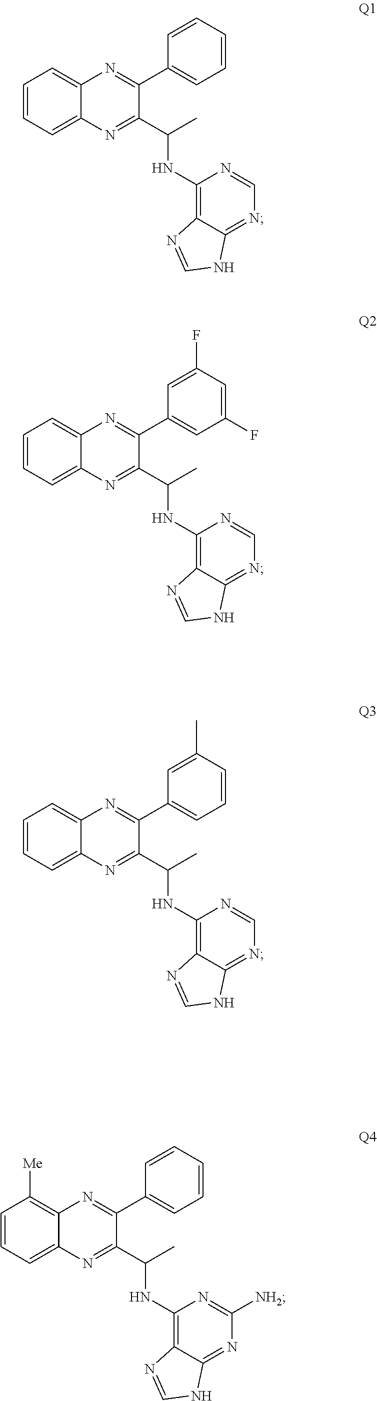 [SMILES: CC(Nc1ncnc2[nH]cnc12)c1nc2ccccc2nc1-c1ccccc1 ; CC(Nc1ncnc2[nH]cnc12)c1nc2ccccc2nc1-c1cc(F)cc(F)c1 ; Cc1cccc(-c2nc3ccccc3nc2C(C)Nc2ncnc3[nH]cnc23)c1 ; Cc1cccc2nc(C(C)Nc3nc(N)nc4[nH]cnc34)c(-c3ccccc3)nc12] 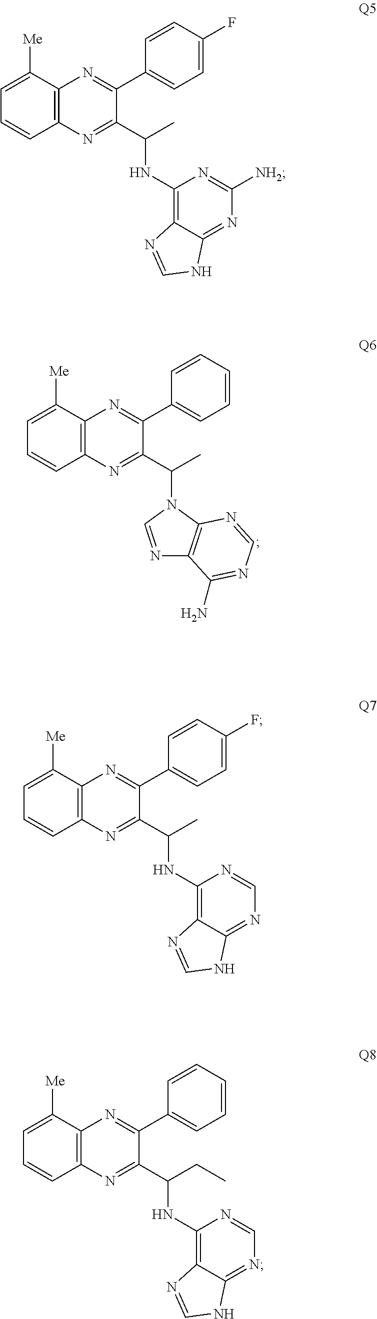 [SMILES: Cc1cccc2nc(C(C)Nc3nc(N)nc4[nH]cnc34)c(-c3ccc(F)cc3)nc12 ; Cc1cccc2nc(C(C)n3cnc4c(N)ncnc43)c(-c3ccccc3)nc12 ; Cc1cccc2nc(C(C)Nc3ncnc4[nH]cnc34)c(-c3ccc(F)cc3)nc12 ; CCC(Nc1ncnc2[nH]cnc12)c1nc2cccc(C)c2nc1-c1ccccc1] 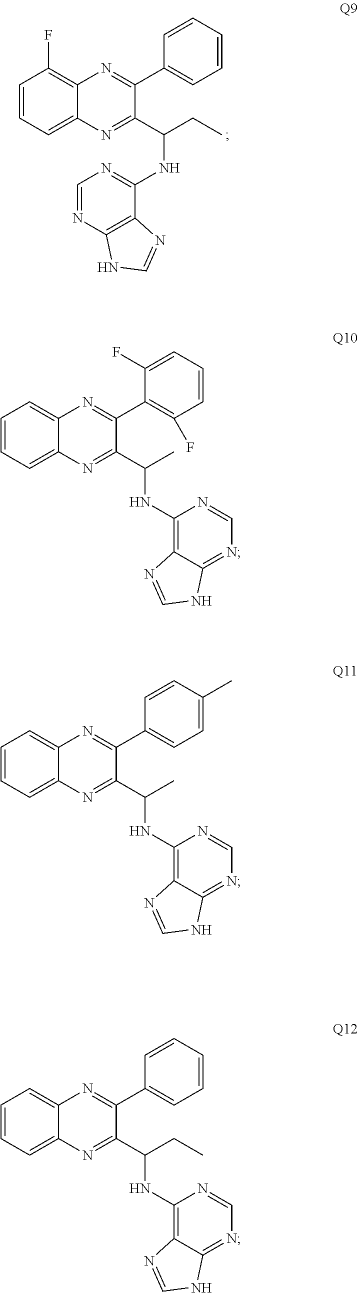 [SMILES: CCC(Nc1ncnc2[nH]cnc12)c1nc2cccc(F)c2nc1-c1ccccc1 ; CC(Nc1ncnc2[nH]cnc12)c1nc2ccccc2nc1-c1c(F)cccc1F ; Cc1ccc(-c2nc3ccccc3nc2C(C)Nc2ncnc3[nH]cnc23)cc1 ; CCC(Nc1ncnc2[nH]cnc12)c1nc2ccccc2nc1-c1ccccc1] 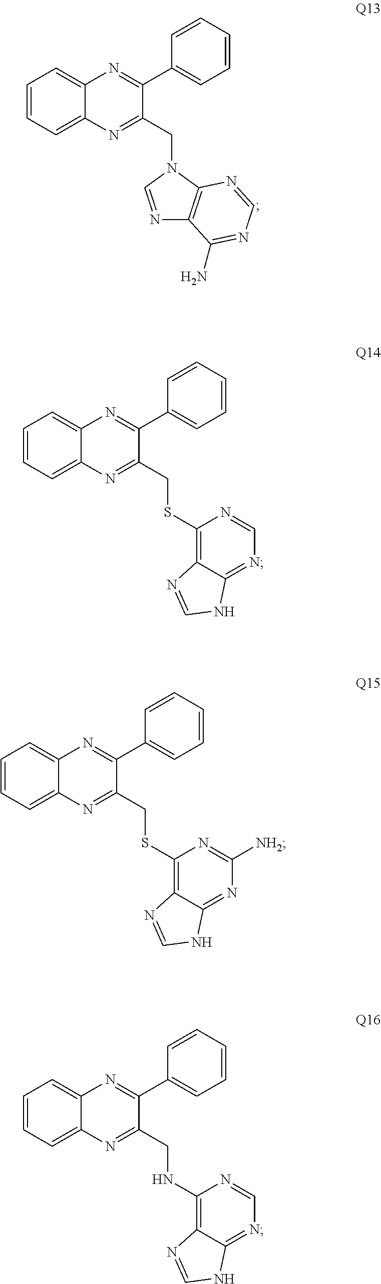 [SMILES: Nc1ncnc2c1ncn2Cc1nc2ccccc2nc1-c1ccccc1 ; c1ccc(-c2nc3ccccc3nc2CSc2ncnc3[nH]cnc23)cc1 ; Nc1nc(SCc2nc3ccccc3nc2-c2ccccc2)c2nc[nH]c2n1 ; c1ccc(-c2nc3ccccc3nc2CNc2ncnc3[nH]cnc23)cc1] 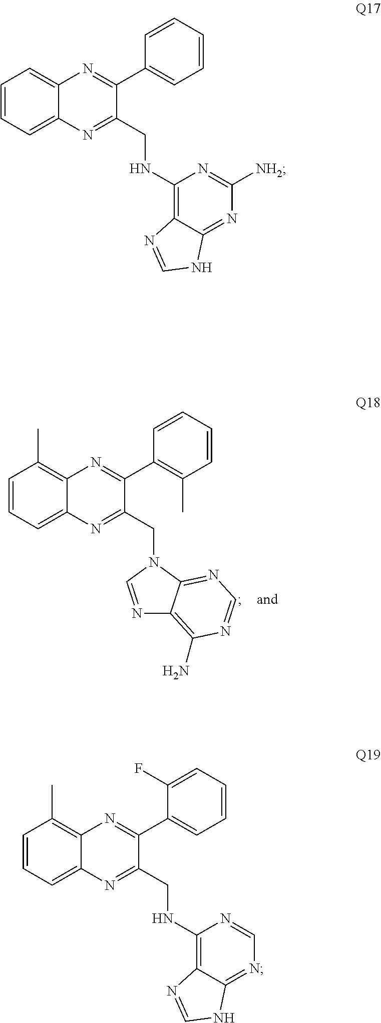 [SMILES: Nc1nc(NCc2nc3ccccc3nc2-c2ccccc2)c2nc[nH]c2n1 ; Cc1ccccc1-c1nc2c(C)cccc2nc1Cn1cnc2c(N)ncnc21 ; Cc1cccc2nc(CNc3ncnc4[nH]cnc34)c(-c3ccccc3F)nc12]
and pharmaceutically acceptable salts thereof.

17. The compound according to claim 16, wherein the compound contains a chiral center contained in the linking group located between the quinoxalinyl and purinyl group; and wherein the chiral center is the S-enantiomer.

18. A method to prevent or treat a condition in a subject in need thereof, wherein said condition is an inflammatory condition or cancer, comprising administering to the subject a therapeutically effective amount of a compound according to claim 1.

19. The method according to claim 18, wherein the condition is an inflammatory condition, and wherein the inflammatory condition is selected from the group consisting of arthritic diseases, ophthalmic disorders, autoimmune diseases, transplant rejection disorders, and inflammatory bowel diseases.

20. The method according to claim 18, wherein the condition is an inflammatory condition, wherein the inflammatory condition is selected from the group consisting of rheumatoid arthritis, psoriatic arthritis, monoarticular arthritis, osteoarthritis, gouty arthritis, spondylitis, Behçet disease, sepsis, septic shock, endotoxic shock, gram negative sepsis, gram positive sepsis, and toxic shock syndrome, multiple organ injury syndrome secondary to septicemia, trauma, or hemorrhage, allergic conjunctivitis, vernal conjunctivitis, uveitis, thyroid-associated ophthalmopathy, eosinophilic granuloma, asthma, chronic bronchitis, allergic rhinitis, acute respiratory distress syndrome (ARDS), chronic obstructive pulmonary disease (COPD), silicosis, pulmonary sarcoidosis, pleurisy, alveolitis, vasculitis, emphysema, pneumonia, bronchiectasis, pulmonary oxygen toxicity, reperfusion injury of the myocardium, brain, or extremities, cystic fibrosis, keloid formation, scar tissue formation, atherosclerosis, systemic lupus erythematosus (SLE), autoimmune thyroiditis, multiple sclerosis, diabetes, Reynaud's syndrome, graft-versus-host-disease (GVHD), allograft rejection, chronic glomerulonephritis, chronic inflammatory bowel disease (CIBD), Crohn's disease, ulcerative colitis, necrotizing enterocolitis, contact dermatitis, atopic dermatitis, psoriasis, or urticaria, fever, myalgias due to infection, meningitis, encephalitis, brain or spinal cord injury due to minor trauma, Sjogren's syndrome, diseases involving leukocyte diapedesis, alcoholic hepatitis, bacterial pneumonia, antigen-antibody complex mediated diseases, hypovolemic shock, Type I diabetes mellitus, acute and delayed hypersensitivity, disease states due to leukocyte dyscrasia and metastasis, thermal injury, granulocyte transfusion-associated syndromes, and cytokine-induced toxicity.

21. The method according to claim 18, wherein the condition is cancer; and wherein said cancer is a hematological malignancy or a solid tumor.

22. The method according to claim 21, wherein the cancer is a hematological malignancy; and said hematological malignancy is selected from the group consisting of acute lymphocytic leukemia (ALL), acute myeloid leukemia (AML), chronic lymphocytic leukemia (CLL), multiple myeloma (MM), and non-Hodgkin lymphoma (NHL). In certain embodiments, the non-Hodgkin lymphoma is selected from the group consisting of large diffuse B-cell lymphoma (LDBCL), mantle cell lymphoma (MCL), Waldenstrom's macroglobulinemia (WM) and lymphoplasmacytic lymphoma.

23. The method according to claim 21, wherein the cancer is a solid tumor; and said solid tumor is selected from the group consisting of myxoid and round cell carcinomas, human soft tissue sarcomas, cancer metastases, squamous cell carcinomas, esophageal squamous cell carcinomas, oral carcinomas, cancers of the adrenal cortex, ACTH-producing tumors, non-small cell lung cancers, breast cancers, gastrointestinal cancers, pancreatic cancers, liver cancers, urological cancers, malignancies of the female reproductive tract, malignancies of the male reproductive tract, kidney cancers, brain cancers, bone cancers, skin cancers, thyroid cancers, retinoblastomas, neuroblastomas, peritoneal effusions, malignant pleural effusions, mesotheliomas, Wilms tumors, gall bladder cancers, trophoblastic neoplasms, hemangiopericytomas, Kaposi's sarcomas, and neuroendocrine cancer.

24. The method according to claim 18, wherein X is C(R^{b})₂ or CH₂CHR^{b}; X has a chiral center; and wherein the chiral center is the S-enantiomer.

25. The method according to claim 1, wherein the compound is selected from the group consisting of 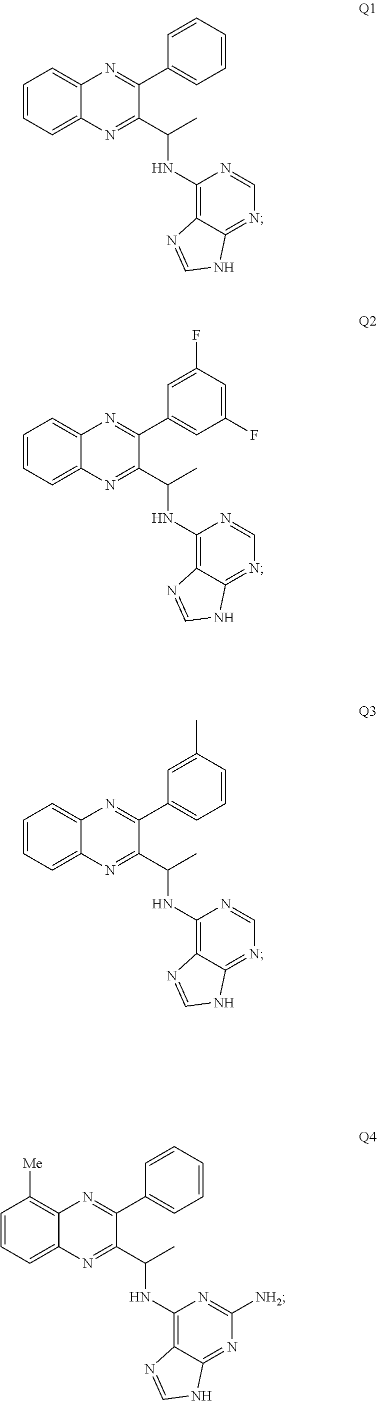 [SMILES: CC(Nc1ncnc2[nH]cnc12)c1nc2ccccc2nc1-c1ccccc1 ; CC(Nc1ncnc2[nH]cnc12)c1nc2ccccc2nc1-c1cc(F)cc(F)c1 ; Cc1cccc(-c2nc3ccccc3nc2C(C)Nc2ncnc3[nH]cnc23)c1 ; Cc1cccc2nc(C(C)Nc3nc(N)nc4[nH]cnc34)c(-c3ccccc3)nc12] 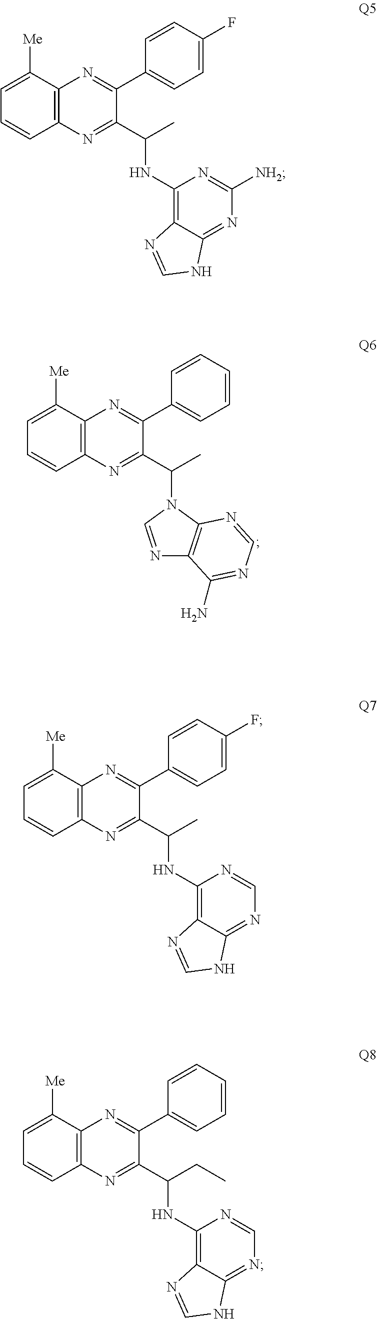 [SMILES: Cc1cccc2nc(C(C)Nc3nc(N)nc4[nH]cnc34)c(-c3ccc(F)cc3)nc12 ; Cc1cccc2nc(C(C)n3cnc4c(N)ncnc43)c(-c3ccccc3)nc12 ; Cc1cccc2nc(C(C)Nc3ncnc4[nH]cnc34)c(-c3ccc(F)cc3)nc12 ; CCC(Nc1ncnc2[nH]cnc12)c1nc2cccc(C)c2nc1-c1ccccc1] 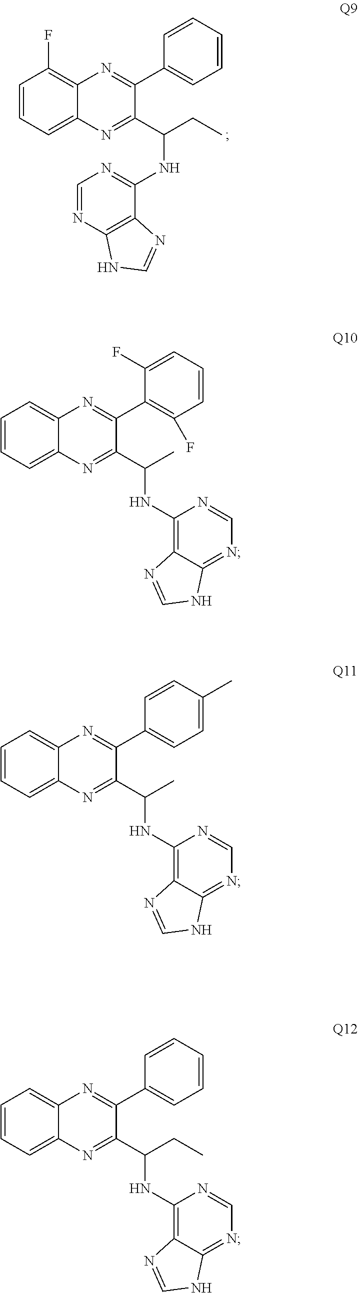 [SMILES: CCC(Nc1ncnc2[nH]cnc12)c1nc2cccc(F)c2nc1-c1ccccc1 ; CC(Nc1ncnc2[nH]cnc12)c1nc2ccccc2nc1-c1c(F)cccc1F ; Cc1ccc(-c2nc3ccccc3nc2C(C)Nc2ncnc3[nH]cnc23)cc1 ; CCC(Nc1ncnc2[nH]cnc12)c1nc2ccccc2nc1-c1ccccc1] 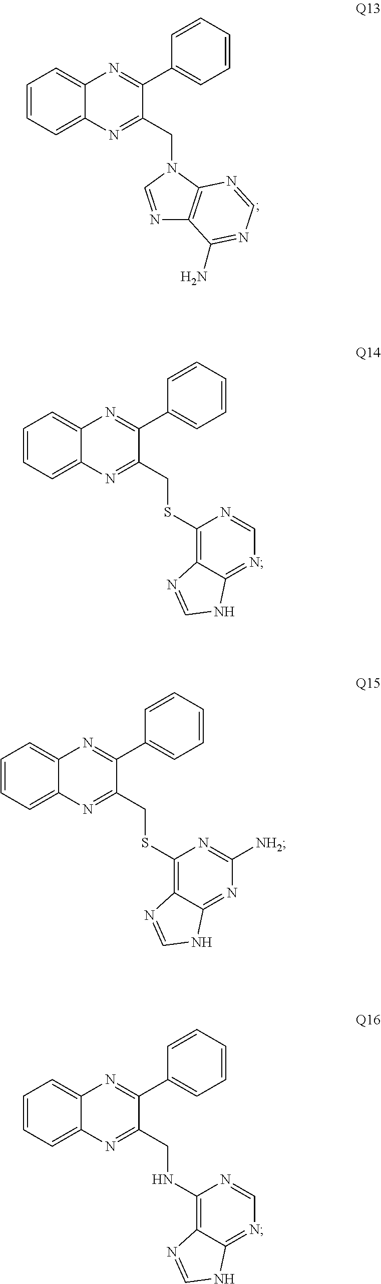 [SMILES: Nc1ncnc2c1ncn2Cc1nc2ccccc2nc1-c1ccccc1 ; c1ccc(-c2nc3ccccc3nc2CSc2ncnc3[nH]cnc23)cc1 ; Nc1nc(SCc2nc3ccccc3nc2-c2ccccc2)c2nc[nH]c2n1 ; c1ccc(-c2nc3ccccc3nc2CNc2ncnc3[nH]cnc23)cc1] 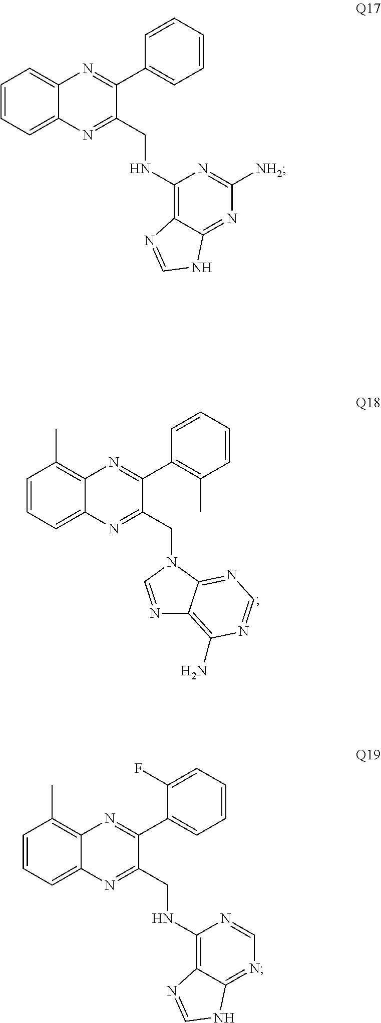 [SMILES: Nc1nc(NCc2nc3ccccc3nc2-c2ccccc2)c2nc[nH]c2n1 ; Cc1ccccc1-c1nc2c(C)cccc2nc1Cn1cnc2c(N)ncnc21 ; Cc1cccc2nc(CNc3ncnc4[nH]cnc34)c(-c3ccccc3F)nc12]
and pharmaceutically acceptable salts thereof.

26. The method according to claim 25, wherein the compound contains a chiral center contained in the linking group located between the quinoxalinyl and purinyl group; and wherein the chiral center is the S-enantiomer.

27. A pharmaceutical composition comprising a compound according to claim 1 or a pharmaceutically acceptable salt thereof; and at least one pharmaceutically acceptable excipient.

28. The pharmaceutical composition according to claim 27, wherein the compound contains a chiral center in the noncyclic linking group between the quinoxaline moiety and the purine moiety.

29. The pharmaceutical composition according to claim 28, wherein the S-enantiomer predominates over the R-enantiomer by a ratio of at least about 9:1
